(19) United States
(12) Patent Application Publication
(10) Pub. No.: US 2025/0195151 A1
(43) Pub. Date: Jun. 19, 2025

(54) Tracker For Magnetically Coupling To Robotic Guide Tube

(71) Applicant: MAKO Surgical Corp., Weston, FL (US)

(72) Inventors: Lucas Gsellman, Medina, OH (US); Victor Soto, Coral Gables, FL (US)

(73) Assignee: MAKO Surgical Corp., Weston, FL (US)

(21) Appl. No.: 18/985,517

(22) Filed: Dec. 18, 2024

Related U.S. Application Data
(60) Provisional application No. 63/612,011, filed on Dec. 19, 2023.

(51) Int. Cl. A61B 34/20; A61B 17/00; A61B 17/34; A61B 34/30

(52) U.S. Cl. CPC A61B 34/20; A61B 17/3423; A61B 2017/00424; A61B 2017/00477; A61B 2017/00876; A61B 2034/2072; A61B 2034/302

(57) ABSTRACT

A surgical system is provided. The surgical system includes a robotic manipulator supporting a guide tube and an instrument configured to be temporarily fixed to the guide tube through magnetic coupling. The instrument supports a tracker.

### CROSS-REFERENCE TO RELATED APPLICATIONS

This application claims priority to U.S. Provisional Application No. 63/612,011, filed Dec. 19, 2023, the entire contents of each of which are incorporated herein by reference.

### BACKGROUND

Robotic surgical systems, such as the MAKO® surgical robot, include a robotic arm that is supported by a base. An end effector is attached to the robotic arm. A base tracker is attached to the base of the robot and positionable via an adjustable support arm. The base tracker is detectable by a camera of a navigation system to track the position of the robot base.

“Robot registration” is a procedure for establishing a relationship between the base tracker and the base of the surgical robot. Robot registration is required, in part, due to the base tracker being adjustably set in any number of poses. Hence, the relationship between the base tracker and the base of the robot is unknown to the navigation system and must be determined using this procedure. In turn, this procedure confirms the accuracy of the robotic arm and the location of the cutting tool supported by the robotic arm, relative to the navigation system.

Conventionally, robot registration involves parking the robot near the surgical site. A registration tool with a shaft is then inserted into the end effector of the robot and is locked to the end effector using a locking mechanism of the end effector. A tracker is then temporarily attached to the shaft of the registration tool. The user is prompted to move the robot, including the registration tool and tracker, between vertices of a predefined cube to facilitate point collection from both the kinematics of the robot arm and tracker. These points are matched and compared throughout the robot registration process.

For certain procedures, such as spinal procedures, robotic manipulators often support a guide tube which has a channel into which various tools are inserted. The guide tube is a passive tube, and unlike the described end effector, does not include any actuatable locking mechanism for securing tools to the guide tube. Hence, the conventional registration tool and tracker used for robot registration are not adapted for use with the guide tube since the guide tube has no means to hold the conventional registration tool and tracker to accurately perform robot registration. Furthermore, outfitting the guide tube with such a locking mechanism would introduce complexity and cost to the system and would significantly increase the footprint of the guide tube, thereby interfering with the surgical procedure.

### SUMMARY OF THE INVENTION

In a first aspect, a surgical system is provided. The surgical system comprises a robotic manipulator supporting a guide tube; and an instrument configured to be temporarily fixed to the guide tube through magnetic coupling and the instrument configured to support a tracker.

In a second aspect, a surgical instrument is provided for use with a robotic manipulator that supports a guide tube. The surgical instrument comprises a body configured to be temporarily fixed to the guide tube through magnetic coupling and the body configured to support a tracker.

In a third aspect, a surgical system is provided. The surgical system comprises a localizer comprising a localizer coordinate system; a robotic manipulator supporting a guide tube; an instrument configured to be temporarily fixed to the guide tube through magnetic coupling, wherein the instrument supports a tracker that is detectable by the localizer; and one or more controllers coupled to the localizer and the robotic manipulator and being configured to facilitate control of the robotic manipulator to move the tracker in various positions to register the robotic manipulator to the localizer coordinate system.

In a fourth aspect, a method is provided of registering a robotic manipulator to a localizer coordinate system of a localizer, the robotic manipulator supporting a guide tube. The method comprises: temporarily fixing an instrument to the guide tube through magnetic coupling, wherein the instrument is configured to support a tracker detectable by the localizer; and facilitating control of the robotic manipulator for moving the tracker in various positions and for registering the robotic manipulator to the localizer coordinate system.

In a fifth aspect, a surgical system is provided. The surgical system comprises: a localizer comprising a localizer coordinate system; a robotic manipulator supporting a guide tube and comprising a manipulator coordinate system; an instrument configured to be temporarily fixed to the guide tube through magnetic coupling, wherein the instrument is configured to support a tracker; and one or more controllers coupled to the localizer and the robotic manipulator and configured to: facilitate control of the robotic manipulator to move the tracker in various positions; obtain, from the localizer, tracking data related to the tracker in the various poses; obtain, from the robotic manipulator, kinematic data related to the robotic manipulator in the various poses; and compare the tracking data and the kinematic data to define a relationship between the manipulator coordinate system and the localizer coordinate system.

In a sixth aspect, a method of operating a surgical system is priovded, the surgical system comprising a localizer comprising a localizer coordinate system, a robotic manipulator supporting a guide tube and comprising a manipulator coordinate system, and an instrument configured to support a tracker. The method comprises: temporarily fixing the instrument to the guide tube through magnetic coupling; facilitating control of the robotic manipulator for moving the tracker in various poses; obtaining, from the localizer, tracking data related to the tracker in the various poses; obtaining, from the robotic manipulator, kinematic data related to the robotic manipulator in the various poses; and comparing the tracking data and the kinematic data for defining a relationship between the manipulator coordinate system and the localizer coordinate system.

In a seventh aspect, a surgical system is provided. The surgical system comprising: a robotic manipulator supporting a guide tube; and a tracker configured to be magnetically attached to the guide tube.

In an eighth aspect, a tracker is provided for use with a robotic manipulator that supports a guide tube. The tracker comprising a body supporting tracking elements and configured to magnetically attach to the guide tube.

In a ninth aspect, a surgical system is provided. The surgical system comprising: a robotic manipulator supporting a guide tube; an instrument configured to generate a magnetic flux field such that the instrument is configured to be temporarily fixed to the guide tube through magnetic coupling, wherein the instrument includes non-ferrous material configured to direct the magnetic flux field; and a tracker configured to be supported by the instrument.

In a tenth aspect, a surgical system is provided. The surgical system comprising: a robotic manipulator supporting a guide tube, the guide tube being configured to receive a surgical tool configured to manipulate anatomy of a patient; and an instrument configured to be temporarily fixed to the guide tube through magnetic coupling; and a tracker configured to be supported by the instrument.

In some implementations, the instrument is configured to be inserted through the guide tube. In some implementations, the guide tube includes a top end and bottom end; the guide tube defines a channel extending between the top end and the bottom end; and the instrument comprises a body configured to be inserted into the channel when the instrument is temporarily fixed to the guide tube through magnetic coupling. In some implementations, the body is configured to substantially occupy the channel when the instrument is temporarily fixed to the guide tube through magnetic coupling. In some implementations, the channel is defined by a guide tube wall that extends between the top end and the bottom end, and wherein the guide tube wall entirely surrounds the channel.

In some implementations, the body comprises a flange configured to abut either the top end or the bottom end of the guide tube when the instrument is temporarily fixed to the guide tube through magnetic coupling. In some implementations, the tracker is coupled directly to the body.

In some implementations, the instrument includes a grip portion coupled to the body and being configured to extend from the guide tube when the instrument is temporarily fixed to the guide tube through magnetic coupling. In some implementations, the grip portion defines the flange. In some implementations, the grip portion is configured to be manually rotated; and the tracker is configured to rotate with rotation of the grip portion.

In some implementations, the body comprises magnetic material configured to magnetically couple to the guide tube. In some implementations, the instrument is configured to be inserted into the channel through the top end of the guide tube, and wherein the flange is configured to abut and rest upon the top end of the guide tube when the instrument is temporarily fixed to the guide tube through magnetic coupling. In some implementations, the instrument is configured to be inserted into the channel through the bottom end of the guide tube, and wherein the flange is configured to abut the bottom end of the guide tube when the instrument is temporarily fixed to the guide tube through magnetic coupling.

In some implementations, the flange comprises magnetic material configured to magnetically couple the flange to either the top end or the bottom end of the guide tube. In some implementations, the grip portion comprises magnetic material configured to magnetically couple the grip portion to either the top end or the bottom end of the guide tube. In some implementations, the instrument is configured to be inserted into the channel through the top end of the guide tube, and wherein the grip portion is configured to extend from the top end of the guide tube when the instrument is temporarily fixed to the guide tube through magnetic coupling. In some implementations, the instrument is configured to be inserted into the channel through the bottom end of the guide tube, and wherein the grip portion is configured to extend from the bottom end of the guide tube when the instrument is temporarily fixed to the guide tube through magnetic coupling.

In some implementations, the instrument comprises a shaft that extends from the body, and wherein the tracker is configured to be supported by the shaft. In some implementations, the tracker is configured to be removably attached to the shaft; and the tracker comprises a coupling interface configured to be installed onto and secured to the shaft. In some implementations, the shaft includes a reference tip located at a distal end of the shaft; and the coupling interface comprises a reference surface configured to abut the reference tip of the shaft. In some implementations, the coupling interface supports a securing mechanism disposed perpendicular to an axis of the shaft and the securing mechanism is configured to be manipulated to apply force to the shaft to secure the tracker to the shaft. In some implementations, the tracker is integrally fixed to the shaft.

### BRIEF DESCRIPTION OF THE DRAWINGS

Other advantages of the present disclosure will be readily appreciated, as the same becomes better understood by reference to the following detailed description when considered in connection with the accompanying drawings.

FIG. 1 is a perspective view of a robotic surgical system including an example registration instrument and a robotic manipulator including an arm and a tool holder.

FIG. 2 is a block diagram of controllers of the robotic surgical system of FIG. 1.

FIG. 3 is a perspective view of the tool holder of FIG. 1 and a first instance of the registration instrument, wherein the registration instrument includes a grip portion, a flange, and a body.

FIG. 4 is a perspective view of the tool holder of FIG. 1 and the first instance of the registration instrument, wherein the registration instrument is inserted into the tool holder.

FIG. 5A is a perspective view of the first instance of the registration instrument.

FIG. 5B is a cutaway view of the first instance of the registration instrument.

FIG. 5C is a diagrammatic view of the first instance of the registration instrument, wherein the grip portion includes a magnetic material, and wherein a magnetic field and magnetic flux field generated by the magnetic material of the grip portion is illustrated.

FIG. 5D is a diagrammatic view of the first instance of the registration instrument, wherein the body includes a magnetic material, and wherein a magnetic field and magnetic flux field generated by the magnetic material of the body is illustrated.

FIG. 6 is a perspective view of the tool holder of FIG. 1 and a second instance of the registration instrument, wherein the registration instrument includes a grip portion, a flange, and a body.

FIG. 7 is a perspective view of the tool holder of FIG. 1 and the second instance of the registration instrument, wherein the registration instrument is inserted into the tool holder.

FIG. 8A is a perspective view of the second instance of the registration instrument.

FIG. 8B is a cutaway view of the second instance of the registration instrument.

FIG. 8C is a diagrammatic view of the second instance of the registration instrument, wherein the grip portion includes a magnetic material, and wherein a magnetic field and magnetic flux field generated by the magnetic material of the grip portion is illustrated.

FIG. 8D is a diagrammatic view of the second instance of the registration instrument, wherein the body includes a magnetic material, and wherein a magnetic field and magnetic flux field generated by the magnetic material of the body is illustrated.

FIG. 8E is a diagrammatic view of the second instance of the registration instrument, wherein the body and the grip portion include a magnetic material, and wherein a magnetic field and magnetic flux field generated by the magnetic material of the body and grip portion is illustrated.

FIG. 9 is a zoomed-in view of a reference surface of a tracker and a reference tip of a shaft of an example registration instrument, wherein the tracker is supported by the shaft of the example registration instrument.

FIG. 10 is a flowchart of a method of registering the robotic manipulator of FIG. 1.

FIG. 11 is an example graphical user interface for instructing a user of the robotic surgical system of FIG. 1 during registration of the robotic manipulator of FIG. 1.

### DETAILED DESCRIPTION OF THE INVENTION

With reference to the Figures, wherein like numerals indicate like or corresponding parts throughout the several views, a surgical system 10 (hereinafter “system”) and method for operating the system 10 are described herein and shown throughout the accompanying Figures.

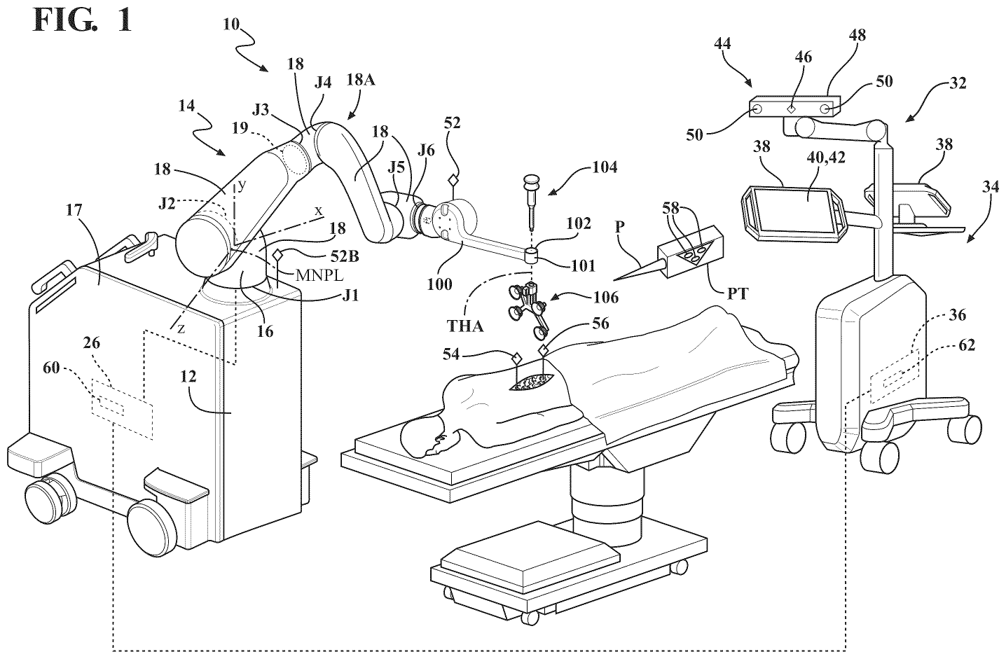

As shown in FIG. 1, the system 10 is a robotic surgical system for treating an anatomy (surgical site) of a patient 12, such as bone or soft tissue. In FIG. 1, the patient 12 is undergoing a surgical procedure. The anatomy in FIG. 1 includes a spine of the patient 12. The surgical procedure may involve tissue removal or treatment. In one aspect, the surgical procedure may involve planning and executing of cannulation of tissue and insertion of an implant within one or more bone structures. In one example, as primarily described herein, the bone structure is a vertebra of the spine. The techniques and advantages described herein, however are not limited only to vertebral bodies, and may be utilized for treating any bone structure, such as those having a cancellous bone region disposed between two cortical bone regions. Such bones may, for example, be in the limbs of the patient, and may include long bones, femurs, pelvic bones, ribs, the skull, or any other bone structure not described herein. The implant can be a pedicle screw when the bone structure is a vertebra. However, other types of implants are contemplated, and the disclosure is not limited solely to pedicle screw preparation.

The system 10 includes a manipulator 14, which may also be referred to as a robotic manipulator. In one example, the manipulator 14 has a base 16 and plurality of links 18. The plurality of links 18 may be commonly referred to as an arm 18A. A manipulator cart 17 supports the manipulator 14 such that the manipulator 14 is fixed to the manipulator cart 17. The links 18 collectively form one or more arms of the manipulator 14. The manipulator 14 may have a serial arm configuration (as shown in FIG. 1) or a parallel arm configuration. In other examples, more than one manipulator 14 may be utilized in a multiple arm configuration. The manipulator 14 comprises a plurality of joints (J) and a plurality of joint encoders 19 located at the joints (J) for determining position data of the joints (J). For simplicity, one joint encoder 19 is illustrated in FIG. 1, although it is to be appreciated that the other joint encoders 19 may be similarly illustrated. The manipulator 14 according to one example has six joints (J1-J6) implementing at least six-degrees of freedom (DOF) for the manipulator 14. However, the manipulator 14 may have any number of degrees of freedom and may have any suitable number of joints (J) and redundant joints (J). In one example, each of the joints (J) of the manipulator 14 are actively driven. In other examples, some joints (J) may be passively driven while other joints (J) are actively driven.

The base 16 of the manipulator 14 is generally a portion of the manipulator 14 that is stationary during usage thereby providing a fixed reference coordinate system (i.e., a virtual zero pose) for other components of the manipulator 14 or the system 10 in general. Generally, the origin of a manipulator coordinate system MNPL is defined at the fixed reference of the base 16. The base 16 may be defined with respect to any suitable portion of the manipulator 14, such as one or more of the links 18. Alternatively, or additionally, the base 16 may be defined with respect to the manipulator cart 17, such as where the manipulator 14 is physically attached to the cart 17. In one example, the base 16 is defined at an intersection of the axes of joints J1 and J2. Thus, although joints J1 and J2 are moving components in reality, the intersection of the axes of joints J1 and J2 is nevertheless a virtual fixed reference point, which does not move in the manipulator coordinate system MNPL. The manipulator 14 and/or manipulator cart 17 house a manipulator computer 26, or other type of control unit.

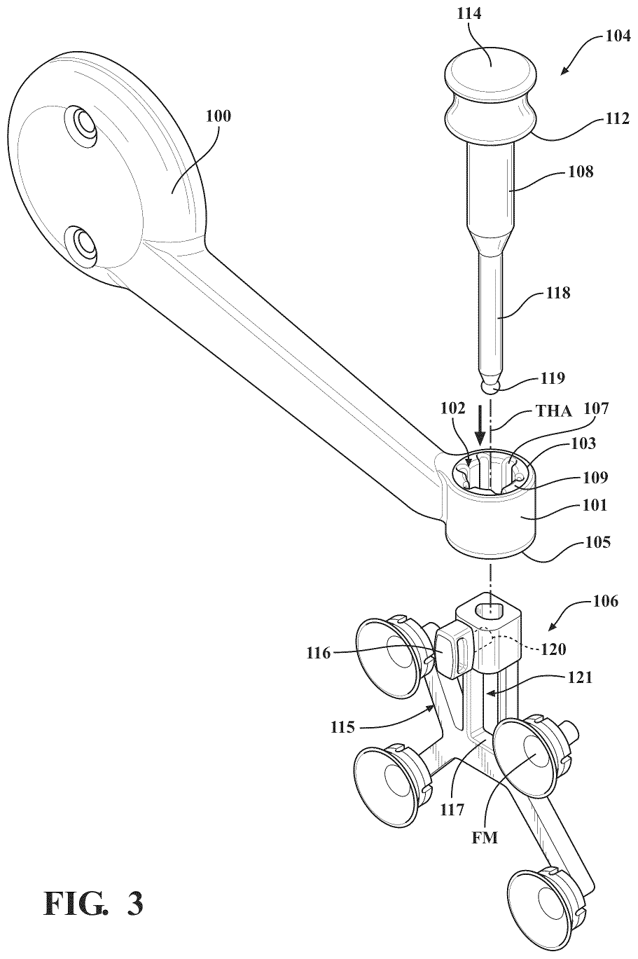

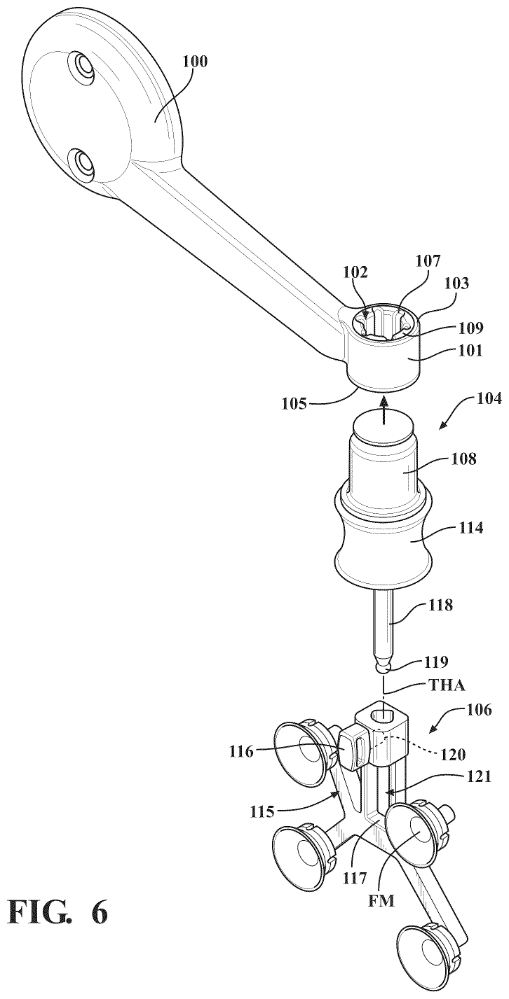

With continued reference to FIG. 1, the manipulator 14 includes the arm 18A and the system 10 includes a tool holder 100 coupled to the arm 18A. The tool holder 100 and the arm 18A may be separate components (i.e., two pieces) or the tool holder 100 and the arm 18A may be integral with one another (i.e., one piece). With reference to FIGS. 3 and 6, the tool holder 100 supports a guide tube 101, which includes a top end 103 and a bottom end 105. The guide tube 101 defines a tool holder channel 102 extending along a tool holder axis THA between the top end 103 and the bottom end 105. The tool holder channel 102 is defined by a guide tube wall 107 that extends between the top end 103 and the bottom end 105.

As shown in FIGS. 3 and 6, the guide tube 101 includes a closed circular shape. The guide tube wall 107 also includes a closed circular shape. The closed shape of the guide tube wall 107 allows the guide tube wall 107 to entirely surround the tool holder channel 102. In other instances, the guide tube wall 107 may include an open shape, such as an open polygonal shape, such that the guide tube wall 107 may not entirely surround the tool holder channel 102.

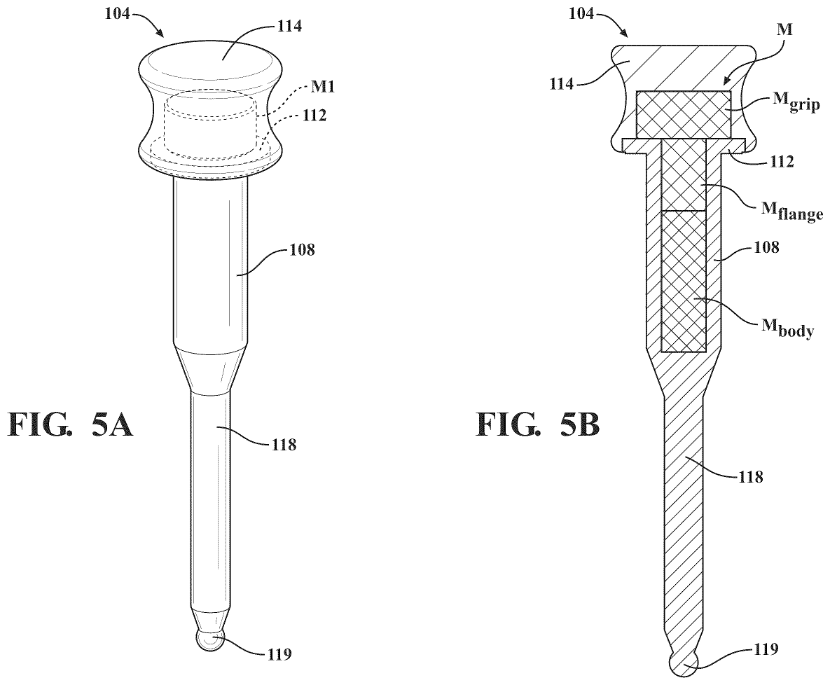

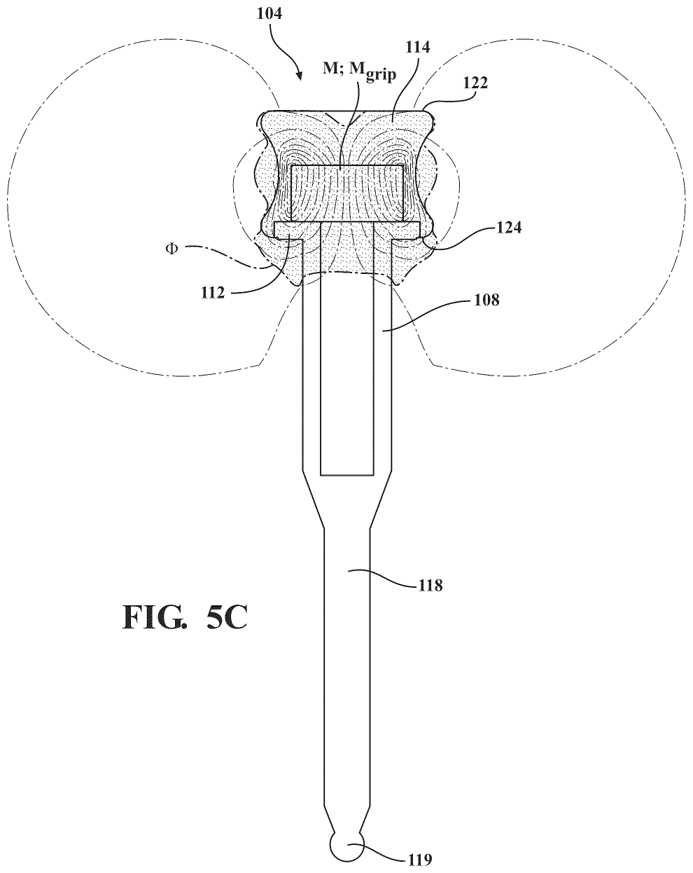

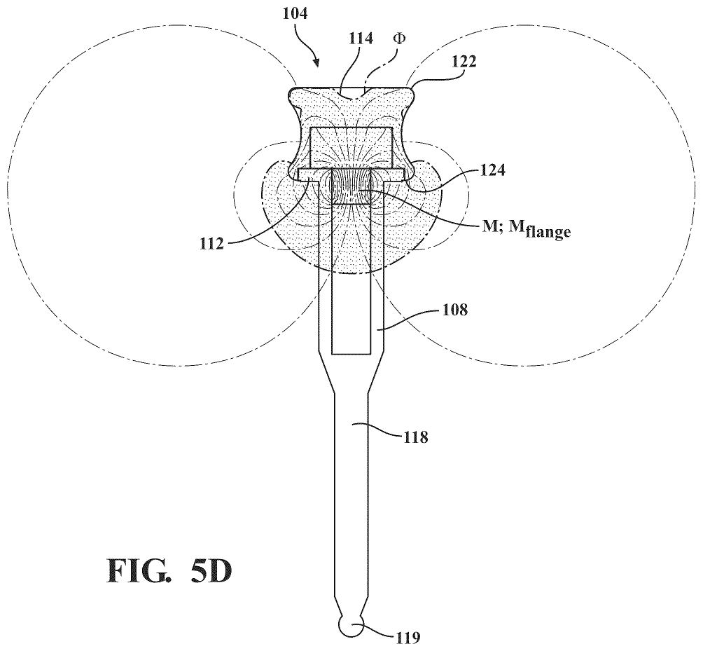

The guide tube 101 and the guide tube wall 107 may include any open or closed shape suitable for constraining one or more degrees of freedom of a tool or registration instrument 104 received by the tool holder channel 102. For example, in the instance of FIGS. 3 and 5, the guide tube 101 and the guide tube wall 107 each include closed circular shapes for constraining four degrees of freedom of a tool or registration instrument 104, while allowing axial and rotational movement therebetween. In an alternate instance, the guide tube 101 and the guide tube wall 107 each may include open circular shapes for constraining four degrees of movement of a tool or registration instrument 104 received by the tool holder channel 102, while allowing axial and rotational movement therebetween. In another alternate instance, the guide tube 101 and the guide tube wall 107 may each include closed polygonal shapes to constrain four degrees of movement of a tool or registration instrument 104 received by the tool holder channel 102, while allowing axial and rotational movement therebetween. In yet another alternate instance, the guide tube 101 may include an open polygonal shape and the guide tube wall 107 may include an open circular shape to constrain four degrees of movement of a tool or registration instrument 104 received by the tool holder channel 102, while allowing axial and rotational movement therebetween.

As shown in FIGS. 3 and 6, the guide tube wall 107 includes six lobes 109 for contacting and constraining one or more degrees of freedom of a tool or registration instrument 104 received by the tool holder channel 102. The guide tube wall 107 may include any suitable number of lobes 109 for contacting and constraining one or more degrees of freedom of a tool or registration instrument 104 received by the tool holder channel 102. In other instances, the guide tube wall 107 may omit the lobes 109. In such instances, the guide tube wall 107 may extend from the guide tube 101 to contact and constrain one or more degrees of freedom of a tool or registration instrument 104 received by the tool holder channel 102.

The guide tube wall 107 may include alternate or additional components for contacting and constraining one or more degrees of freedom of a tool or registration instrument 104 received by the tool holder channel 102. For example, the guide tube wall 107 may alternatively or additionally include slits extending from the top end 103 to the bottom end 105 for receiving surgical tools, such as a scalpel.

During registration of the system 10, the guide tube 101 may be temporarily fixed to a registration instrument 104 through magnetic coupling. The registration instrument 104 may support a tracker 106, which includes fiducial markers FM that may be tracked by a navigation system 32 of the system 10 during registration. For example, in the instances of FIGS. 3 and 6, the tracker 106 includes four fiducial markers FM. A method of registering the system 10 while the guide tube 101 is temporarily fixed to the registration instrument 104 and the tracker 106 is supported by the registration instrument 104 will be described in greater detail herein.

The guide tube 101 is configured to receive a surgical tool for performing a surgical procedure. For example, the surgical tool may be any surgical tool for manipulating the anatomy of the patient 12, such as a tap, probe, drill, dilator, or screwdriver. The surgical tool may be inserted into the channel 102 of the tool holder such that the guide tube 101 can assist with holding the surgical tool on the tool holder axis THA of the guide tube 101. Additionally, the robotic manipulator 14 may be configured to align the tool holder 100 such that the tool holder axis THA is aligned with a target axis defined relative to the vertebral body (or other anatomical part) to perform the surgical procedure. Such a configuration of the tool holder 100 is further described in U.S. Provisional Patent Application No. 63/454,346, entitled, “Anti-Skiving Guide Tube and Surgical System Including the Same”, which is incorporated herein by reference.

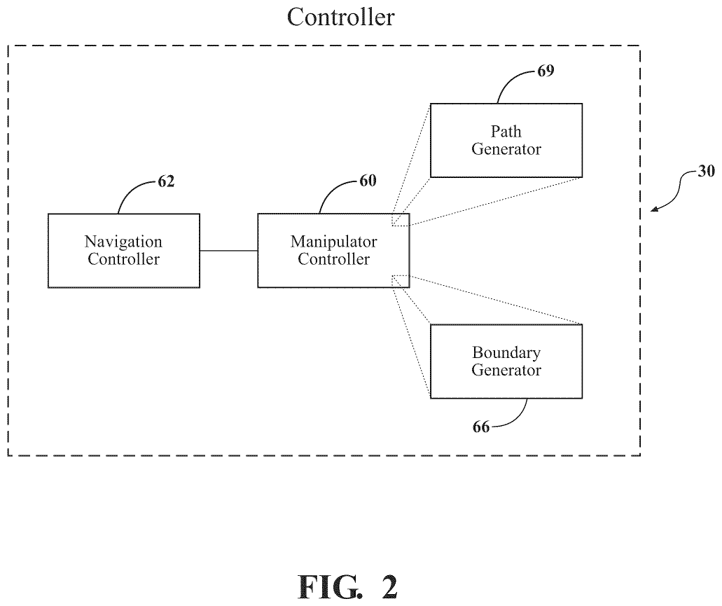

Referring to FIG. 2, the system 10 includes one or more controllers 30 (hereinafter referred to as “controller”). The controller 30 includes software and/or hardware for controlling the manipulator 14. The controller 30 directs the motion of the manipulator 14 and controls a state (position and/or orientation) of the tool holder 100 with respect to a coordinate system. In one example, the coordinate system is the manipulator coordinate system MNPL, as shown in FIG. 1. The manipulator coordinate system MNPL has an origin located at any suitable pose with respect to the manipulator 14. Axes of the manipulator coordinate system MNPL may be arbitrarily chosen as well. Generally, the origin of the manipulator coordinate system MNPL is defined at the fixed reference point of the base 16. One example of the manipulator coordinate system MNPL is described in U.S. Pat. No. 9,119,655, entitled, “Surgical Manipulator Capable of Controlling a Surgical Instrument in Multiple Modes,” the disclosure of which is hereby incorporated by reference.

As shown in FIG. 1, the system 10 further includes a navigation system 32. One example of the navigation system 32 is described in U.S. Pat. No. 9,008,757, filed on Sep. 24, 2013, entitled, “Navigation System Including Optical and Non-Optical Sensors,” hereby incorporated by reference. The navigation system 32 is configured to track movement of various objects. Such objects include, for example, the manipulator 14, the tool holder 100, guide tube 101, the surgical tool, and/or the anatomy, e.g., certain vertebrae or the pelvis of the patient. The navigation system 32 tracks these objects to gather state information of one or more of the objects with respect to a (navigation) localizer coordinate system LCLZ. Coordinates in the localizer coordinate system LCLZ may be transformed to the manipulator coordinate system MNPL, and/or vice-versa, using transformation techniques described herein.

The navigation system 32 includes a cart assembly 34 that houses a navigation computer 36, and/or other types of control units. A navigation interface is in operative communication with the navigation computer 36. The navigation interface includes one or more displays 38. The navigation system 32 is capable of displaying a graphical representation of the relative states of the tracked objects to the operator using the one or more displays 38. First and second input devices 40, 42 may be used to input information into the navigation computer 36 or otherwise to select/control certain aspects of the navigation computer 36. As shown in FIG. 1, such input devices 40, 42 include interactive touchscreen displays. However, the input devices 40, 42 may include any one or more of a keyboard, a mouse, a microphone (voice-activation), gesture control devices, head-mounted devices, and the like.

The navigation system 32 is configured to depict a visual representation of the anatomy and the tool holder 100, guide tube 101, and/or surgical tool for visual guidance of any of the techniques described. The visual representation may be real (camera) images, virtual representations (e.g., computer models), or any combination thereof. The visual representation can be presented on any display viewable to the surgeon, such as the displays 38 of the navigation system 32, head mounted devices, or the like. The representations may be augmented reality, mixed reality, or virtual reality.

The navigation system 32 also includes a navigation localizer 44 (hereinafter “localizer”) coupled to the navigation computer 36. In one example, the localizer 44 is an optical localizer and includes a camera unit 46. The camera unit 46 has an outer casing 48 that houses one or more optical sensors 50.

The navigation system 32 may include one or more trackers. In one example, the trackers include a pointer tracker PT, one or more manipulator trackers 52, one or more patient trackers 54, 56. In the illustrated example of FIG. 1, the manipulator tracker 52A is attached to the tool holder 100, the first patient tracker 54 is firmly affixed to a vertebra of the patient 12, and the second patient tracker 56 is firmly affixed to pelvis of the patient 12. In this example, the patient trackers 54, 56 are firmly affixed to sections of bone. The pointer tracker PT is firmly affixed to a pointer P used for registering the anatomy to the localizer coordinate system LCLZ. The manipulator tracker 52 may be affixed to any suitable component of the manipulator 14, in addition to, or other than the tool holder 100, guide tube 101, and/or surgical tool, such as the base 16 (i.e., tracker 52B), or any one or more links 18 of the manipulator 14. Those skilled in the art appreciate that the trackers 52, 54, 56, PT may be fixed to their respective components in any suitable manner.

In one example, the base tracker 52B is attached to one end of an adjustable support arm. The adjustable support arm is attached at the other end to the cart 17 and the adjustable support arm can be positioned and locked to place the base tracker 52B in a fixed position relative to the cart 17. An example of a base tracker 52B coupled to the adjustable support arm can be like that described in U.S. patent application Ser. No. 17/513,324, entitled, “Robotic Surgical System With Motorized Movement To A Starting Pose For A Registration Or Calibration Routine”, the entire contents of which is hereby incorporated by reference in its entirety. In another example, the base tracker 52B may include a plurality of (active or passive) tracking elements located on any number of links 18 of the manipulator 14. In this case, the base tracker 52B is formed of a tracking geometry from the various tracking elements, which move with movement of the robotic arm. An example of a base tracker 52B formed by optical markers located on the links 18 may be like that described in U.S. patent application Ser. No. 18/115,964, entitled, “Robotic System with Link Tracker”, the entire contents of which is hereby incorporated by reference in its entirety.

When optical localization is utilized, however, one or more of the trackers may include active markers 58. The active markers 58 may include light emitting diodes (LEDs). Alternatively, the trackers 52, 54, 56 may have passive markers, such as reflectors, which reflect light emitted from the camera unit 46. Other suitable markers not specifically described herein may be utilized.

The localizer 44 tracks the trackers 52, 54, 56 to determine a state of one or more of the trackers 52, 54, 56, which correspond respectively to the state of the object respectively attached thereto. The localizer 44 provides the state of the trackers 52, 54, 56 to the navigation computer 36. In one example, the navigation computer 36 determines and communicates the state the trackers 52, 54, 56 to the manipulator computer 26. As used herein, the state of an object includes, but is not limited to, data that defines the position and/or orientation of the tracked object or equivalents/derivatives of the position and/or orientation. For example, the state may be a pose of the object, and may include linear data, and/or angular velocity data, and the like.

Although one example of the navigation system 32 is shown in the Figures, the navigation system 32 may have any other suitable configuration for tracking the manipulator 14 and the patient 12. The illustrated tracker configuration is provided merely as one example for tracking objects within the operating space. Any number of trackers may be utilized and may be located in positions or on objects other than shown. In other examples, such as described below, the localizer 44 may detect objects absent any trackers affixed to objects.

In one example, the navigation system 32 and/or localizer 44 are ultrasound-based. For example, the navigation system 32 may comprise an ultrasound imaging device coupled to the navigation computer 36. The ultrasound imaging device may be robotically controlled or may be hand-held. The ultrasound imaging device images any of the aforementioned objects, e.g., the manipulator 14 and the patient 12, and generates state signals to the controller 30 based on the ultrasound images. The ultrasound images may be of any ultrasound imaging modality. The navigation computer 36 may process the images in near real-time to determine states of the objects. Ultrasound tracking can be performed absent the use of trackers affixed to the objects being tracked. The ultrasound imaging device may have any suitable configuration and may be different than the camera unit 46 as shown in FIG. 1. One example of an ultrasound tracking system can be like that described in U.S. patent application Ser. No. 15/999,152, filed Aug. 16, 2018, entitled “Ultrasound Bone Registration With Learning-Based Segmentation And Sound Speed Calibration,” the entire contents of which are incorporated by reference herein.

In another example, the navigation system 32 and/or localizer 44 are radio frequency (RF)-based. For example, the navigation system 32 may comprise an RF transceiver coupled to the navigation computer 36. The manipulator 14 and the patient 12 may comprise RF emitters or transponders attached thereto. The RF emitters or transponders may be passive or actively energized. The RF transceiver transmits an RF tracking signal and generates state signals to the controller 30 based on RF signals received from the RF emitters. The navigation computer 36 and/or the controller 30 may analyze the received RF signals to associate relative states thereto. The RF signals may be of any suitable frequency. The RF transceiver may be positioned at any suitable location to track the objects using RF signals effectively. Furthermore, the RF emitters or transponders may have any suitable structural configuration that may be much different than the trackers 52, 54, 56 as shown in FIG. 1.

In yet another example, the navigation system 32 and/or localizer 44 are electromagnetically based. For example, the navigation system 32 may comprise an EM transceiver coupled to the navigation computer 36. The manipulator 14 and the patient 12 may comprise EM components attached thereto, such as any suitable magnetic tracker, electro-magnetic tracker, inductive tracker, or the like. The trackers may be passive or actively energized. The EM transceiver generates an EM field and generates state signals to the controller 30 based upon EM signals received from the trackers. The navigation computer 36 and/or the controller 30 may analyze the received EM signals to associate relative states thereto. Again, such navigation system 32 examples may have structural configurations that are different than the navigation system 32 configuration as shown throughout the Figures.

In yet another example, the navigation system 32 and/or localizer 44 utilize a machine vision system which includes a video camera coupled to the navigation computer 36. The video camera is configured to locate a physical object in a target space. The physical object has a geometry represented by virtual object data stored by the navigation computer 36. The detected objects may be tools, obstacles, anatomical features, trackers, or the like. The video camera and navigation computer 36 are configured to detect the physical objects using image processing techniques such as pattern, color, or shape recognition, edge detection, pixel analysis, neutral net or deep learning processing, optical character recognition, barcode detection, or the like. The navigation computer 36 can compare the captured images to the virtual object data to identify and track the objects. A tracker may or may not be coupled to the physical object. If trackers are utilized, the machine vision system may also include infrared detectors for tracking the trackers and comparing tracking data to machine vision data. Again, such navigation system 32 examples may have structural configurations that are different than the navigation system 32 configuration as shown throughout the Figures. Examples of machine vision tracking systems can be like that described in U.S. Pat. No. 9,603,665, entitled “Systems and Methods for Establishing Virtual Constraint Boundaries” and/or like that described in U.S. Provisional Patent Application No. 62/698,502, filed Jul. 16, 2018, entitled “Systems and Method for Image Based Registration and Calibration,” the entire contents of which are incorporated by reference herein.

The navigation system 32 and/or localizer 44 may have any other suitable components or structure not specifically recited herein. Furthermore, any of the techniques, methods, and/or components described above with respect to the camera-based navigation system 32 shown throughout the Figures may be implemented or provided for any of the other examples of the navigation system 32 described herein. For example, the navigation system 32 may utilize solely inertial tracking or any combination of tracking techniques.

As shown in FIG. 2, the controller 30 further includes software modules. The software modules may be part of a computer program or programs that operate on the manipulator computer 26, navigation computer 36, or a combination thereof, to process data to assist with control of the system 10. The software modules include instructions stored in one or more non-transitory computer readable medium or memory on the manipulator computer 26, navigation computer 36, or a combination thereof, to be executed by one or more processors of the computers 26, 36. Additionally, software modules for prompting and/or communicating with the operator may form part of the program or programs and may include instructions stored in memory on the manipulator computer 26, navigation computer 36, or a combination thereof. The operator interacts with the first and second input devices 40, 42 and the one or more displays 38 to communicate with the software modules. The user interface software may run on a separate device from the manipulator computer 26 and navigation computer 36.

The controller 30 includes a manipulator controller 60 for processing data to direct motion of the manipulator 14. In one example, as shown in FIG. 1, the manipulator controller 60 is implemented on the manipulator computer 26. The manipulator controller 60 may receive and process data from a single source or multiple sources. The controller 30 further includes a navigation controller 62 for communicating the state data relating to the anatomy to the manipulator 14 to the manipulator controller 60. The manipulator controller 60 receives and processes the state data provided by the navigation controller 62 to direct movement of the manipulator 14. In one example, as shown in FIG. 1, the navigation controller 62 is implemented on the navigation computer 36. The manipulator controller 60 or navigation controller 62 may also communicate states of the patient 12 and manipulator 14 to the operator by displaying an image of the anatomy and the manipulator 14 on the one or more displays 38. The manipulator computer 26 or navigation computer 36 may also command display of instructions or request information using the display 38 to interact with the operator and for directing the manipulator 14.

The one or more controllers 30, including the manipulator controller 60 and navigation controller 62, may be implemented on any suitable device or devices in the system 10, including, but not limited to, the manipulator computer 26, the navigation computer 36, and any combination thereof. As will be described herein, the controller 30 is not limited to one controller, but may include a plurality of controllers for various systems, components, or sub-systems of the surgical system 10. These controllers may be in communication with each other (e.g., directly, or indirectly), and/or with other components of the surgical system 10, such as via physical electrical connections (e.g., a tethered wire harness) and/or via one or more types of wireless communication (e.g., with a WiFi™ network, Bluetooth®, a radio network, and the like). Any of the one or more controllers 30 may be realized as or with various arrangements of computers, processors, control units, and the like, and may comprise discrete components or may be integrated (e.g., sharing hardware, software, inputs, outputs, and the like). Any of the one or more controllers may implement their respective functionality using hardware-only, software-only, or a combination of hardware and software. Examples of hardware include, but is not limited, single or multi-core processors, CPUs, GPUs, integrated circuits, microchips, or ASICs, digital signal processors, microcontrollers, field programmable gate arrays, systems on a chip, discrete circuitry, and/or other suitable hardware, and the like. The one or more controllers may implement software programs, software modules, algorithms, logical rules, look-up tables and other reference data, and various software layers for implementing any of the capabilities described herein. Equivalents of the software and hardware for the one or more controllers 30, and peripheral devices connected thereto, are fully contemplated.

As shown in FIG. 2, the controller 30 includes a boundary generator 66. The boundary generator 66 is a software module that may be implemented on the manipulator controller 60. Alternatively, the boundary generator 66 may be implemented on other components, such as the navigation controller 62. The boundary generator 66 generates virtual boundaries (VB) for constraining the tool holder 100, guide tube 101, registration instrument 104, and/or a surgical tool. Such virtual boundaries (VB) may also be referred to as virtual meshes, virtual constraints, line haptics, or the like. The virtual boundaries (VB) may be defined with respect to a 3-D bone model registered to the one or more patient trackers 54, 56 such that the virtual boundaries (VB) are fixed relative to the bone model. The state of the tool holder 100, guide tube 101, registration instrument 104, and/or a surgical tool is tracked relative to the virtual boundaries (VB). In one example, the state of the TCP is measured relative to the virtual boundaries (VB) for purposes of determining when and where haptic feedback force is applied to the manipulator 14, or more specifically, the tool 20 and/or bur 24.

A tool path generator 68 is another software module run by the controller 30, and more specifically, the manipulator controller 60. The tool path generator 68 generates a path for the tool holder 100 and/or a surgical tool to traverse, such as for removing sections of the anatomy to receive an implant. One exemplary system and method for generating the tool path is explained in U.S. Pat. No. 9,119,655, entitled, “Surgical Manipulator Capable of Controlling a Surgical Instrument in Multiple Modes,” the disclosure of which is hereby incorporated by reference. In some examples, the virtual boundaries (VB) and/or tool paths may be generated offline rather than on the manipulator computer 26 or navigation computer 36. Thereafter, the virtual boundaries (VB) and/or tool paths may be utilized at runtime by the manipulator controller 60.

Additionally, it may be desirable to control the manipulator 14 in different modes of operation for the system 10. For example, the system 10 may enable the manipulator 14 to interact with the site using manual and semi-autonomous modes of operation. An example of the semi-autonomous mode is described in U.S. Pat. No. 9,119,655, entitled, “Surgical Manipulator Capable of Controlling a Surgical Instrument in Multiple Modes,” the disclosure of which is hereby incorporated by reference. In the semi-autonomous mode, the manipulator 14 directs movement of the tool holder 100 at the surgical site. In one instance, the controller 30 models the tool holder 100 and/or surgical tool as a virtual rigid body and determines forces and torques to apply to the virtual rigid body to advance and constrain the tool holder 100 and/or surgical tool along any trajectory or path in the semi-autonomous mode. Movement of the tool 20 in the semi-autonomous mode is constrained in relation to the virtual constraints generated by the boundary generator 66 and/or path generator 69,

In the semi-autonomous mode, the manipulator 14 is capable of moving the tool holder 100 free of operator assistance. Free of operator assistance may mean that an operator does not physically move the tool holder 100 by applying external force to move the tool holder 100. Instead, the operator may use some form of control to manage starting and stopping of movement. For example, the operator may hold down a button of a control to start movement of the tool holder 100 and release the button to stop movement of the tool holder 100. Alternatively, the operator may press a button to start movement of the tool holder 100 and press a button to stop motorized movement of the tool 20 along the trajectory or path. The manipulator 14 uses motorized movement to advance the tool holder 100 in accordance with pre-planned parameters.

Alternatively, the system 10 may be operated in the manual mode. Here, in one instance, the operator manually directs, and the manipulator 14 controls, movement of the tool holder 100 at the surgical site. The operator physically contacts the tool holder 100 to cause movement of the tool holder 100. The manipulator 14 monitors the forces and torques placed on the tool holder 100 by the operator in order to position the tool holder. A sensor that is part of the manipulator 14, such as a force-torque transducer, measures these external forces and torques applied to the manipulator 14 and/or holder 100, e.g., in six degrees of freedom. In one example, the sensor is coupled between the distal-most link of the manipulator (J6) and the tool holder 100. In response to the applied forces and torques, the one or more controllers 30, 60, 62 are configured to determine a commanded position of the tool holder 100 by evaluating the forces/torques applied externally to the tool holder 100 with respect to virtual model of the tool holder 100 and/or surgical tool in a virtual simulation. The manipulator 14 then mechanically moves the tool holder 100 to the commanded position in a manner that emulates the movement that would have occurred based on the forces and torques applied externally by the operator. Movement of the tool holder 100 in the manual mode is also constrained in relation to the virtual constraints generated by the boundary generator 66 and/or path generator 69.

Referring to FIG. 2, the system 10 includes one or more controllers 30 (hereinafter referred to as “controller”). The controller 30 includes software and/or hardware for controlling the manipulator 14. The controller 30 directs the motion of the manipulator 14 and controls a state (position and/or orientation) of the tool holder 100 with respect to a coordinate system. In one example, the coordinate system is the manipulator coordinate system MNPL, as shown in FIG. 1. The manipulator coordinate system MNPL has an origin located at any suitable pose with respect to the manipulator 14. Axes of the manipulator coordinate system MNPL may be arbitrarily chosen as well. Generally, the origin of the manipulator coordinate system MNPL is defined at the fixed reference point of the base 16. One example of the manipulator coordinate system MNPL is described in U.S. Pat. No. 9,119,655, entitled, “Surgical Manipulator Capable of Controlling a Surgical Instrument in Multiple Modes,” the disclosure of which is hereby incorporated by reference.

As shown in FIG. 1, the system 10 further includes a navigation system 32. One example of the navigation system 32 is described in U.S. Pat. No. 9,008,757, filed on Sep. 24, 2013, entitled, “Navigation System Including Optical and Non-Optical Sensors,” hereby incorporated by reference. The navigation system 32 is configured to track movement of various objects. Such objects include, for example, the manipulator 14, the tool holder 100, guide tube 101, the surgical tool, and/or the anatomy, e.g., certain vertebrae or the pelvis of the patient. The navigation system 32 tracks these objects to gather state information of one or more of the objects with respect to a (navigation) localizer coordinate system LCLZ. Coordinates in the localizer coordinate system LCLZ may be transformed to the manipulator coordinate system MNPL, and/or vice-versa, using transformation techniques described herein.

The navigation system 32 includes a cart assembly 34 that houses a navigation computer 36, and/or other types of control units. A navigation interface is in operative communication with the navigation computer 36. The navigation interface includes one or more displays 38. The navigation system 32 is capable of displaying a graphical representation of the relative states of the tracked objects to the operator using the one or more displays 38. First and second input devices 40, 42 may be used to input information into the navigation computer 36 or otherwise to select/control certain aspects of the navigation computer 36. As shown in FIG. 1, such input devices 40, 42 include interactive touchscreen displays. However, the input devices 40, 42 may include any one or more of a keyboard, a mouse, a microphone (voice-activation), gesture control devices, head-mounted devices, and the like.

The navigation system 32 is configured to depict a visual representation of the anatomy and the tool holder 100, guide tube 101, and/or surgical tool for visual guidance of any of the techniques described. The visual representation may be real (camera) images, virtual representations (e.g., computer models), or any combination thereof. The visual representation can be presented on any display viewable to the surgeon, such as the displays 38 of the navigation system 32, head mounted devices, or the like. The representations may be augmented reality, mixed reality, or virtual reality.

The navigation system 32 also includes a navigation localizer 44 (hereinafter “localizer”) coupled to the navigation computer 36. In one example, the localizer 44 is an optical localizer and includes a camera unit 46. The camera unit 46 has an outer casing 48 that houses one or more optical sensors 50.

The navigation system 32 may include one or more trackers. In one example, the trackers include a pointer tracker PT, one or more manipulator trackers 52, one or more patient trackers 54, 56. In the illustrated example of FIG. 1, the manipulator tracker 52 is attached to the tool holder 100, the first patient tracker 54 is firmly affixed to a vertebra of the patient 12, and the second patient tracker 56 is firmly affixed to pelvis of the patient 12. In this example, the patient trackers 54, 56 are firmly affixed to sections of bone. The pointer tracker PT is firmly affixed to a pointer P used for registering the anatomy to the localizer coordinate system LCLZ. The manipulator tracker 52 may be affixed to any suitable component of the manipulator 14, in addition to, or other than the tool holder 100, guide tube 101, and/or surgical tool, such as the base 16 (i.e., tracker 52B), or any one or more links 18 of the manipulator 14. Those skilled in the art appreciate that the trackers 52, 54, 56, PT may be fixed to their respective components in any suitable manner.

When optical localization is utilized, however, one or more of the trackers may include active markers 58. The active markers 58 may include light emitting diodes (LEDs). Alternatively, the trackers 52, 54, 56 may have passive markers, such as reflectors, which reflect light emitted from the camera unit 46. Other suitable markers not specifically described herein may be utilized.

The localizer 44 tracks the trackers 52, 54, 56 to determine a state of one or more of the trackers 52, 54, 56, which correspond respectively to the state of the object respectively attached thereto. The localizer 44 provides the state of the trackers 52, 54, 56 to the navigation computer 36. In one example, the navigation computer 36 determines and communicates the state the trackers 52, 54, 56 to the manipulator computer 26. As used herein, the state of an object includes, but is not limited to, data that defines the position and/or orientation of the tracked object or equivalents/derivatives of the position and/or orientation. For example, the state may be a pose of the object, and may include linear data, and/or angular velocity data, and the like.

Although one example of the navigation system 32 is shown in the Figures, the navigation system 32 may have any other suitable configuration for tracking the manipulator 14 and the patient 12. The illustrated tracker configuration is provided merely as one example for tracking objects within the operating space. Any number of trackers may be utilized and may be located in positions or on objects other than shown. In other examples, such as described below, the localizer 44 may detect objects absent any trackers affixed to objects.

In one example, the navigation system 32 and/or localizer 44 are ultrasound-based. For example, the navigation system 32 may comprise an ultrasound imaging device coupled to the navigation computer 36. The ultrasound imaging device may be robotically controlled or may be hand-held. The ultrasound imaging device images any of the aforementioned objects, e.g., the manipulator 14 and the patient 12, and generates state signals to the controller 30 based on the ultrasound images. The ultrasound images may be of any ultrasound imaging modality. The navigation computer 36 may process the images in near real-time to determine states of the objects. Ultrasound tracking can be performed absent the use of trackers affixed to the objects being tracked. The ultrasound imaging device may have any suitable configuration and may be different than the camera unit 46 as shown in FIG. 1. One example of an ultrasound tracking system can be like that described in U.S. patent application Ser. No. 15/999,152, filed Aug. 16, 2018, entitled “Ultrasound Bone Registration With Learning-Based Segmentation And Sound Speed Calibration,” the entire contents of which are incorporated by reference herein.

In another example, the navigation system 32 and/or localizer 44 are radio frequency (RF)-based. For example, the navigation system 32 may comprise an RF transceiver coupled to the navigation computer 36. The manipulator 14 and the patient 12 may comprise RF emitters or transponders attached thereto. The RF emitters or transponders may be passive or actively energized. The RF transceiver transmits an RF tracking signal and generates state signals to the controller 30 based on RF signals received from the RF emitters. The navigation computer 36 and/or the controller 30 may analyze the received RF signals to associate relative states thereto. The RF signals may be of any suitable frequency. The RF transceiver may be positioned at any suitable location to track the objects using RF signals effectively. Furthermore, the RF emitters or transponders may have any suitable structural configuration that may be much different than the trackers 52, 54, 56 as shown in FIG. 1.

In yet another example, the navigation system 32 and/or localizer 44 are electromagnetically based. For example, the navigation system 32 may comprise an EM transceiver coupled to the navigation computer 36. The manipulator 14 and the patient 12 may comprise EM components attached thereto, such as any suitable magnetic tracker, electro-magnetic tracker, inductive tracker, or the like. The trackers may be passive or actively energized. The EM transceiver generates an EM field and generates state signals to the controller 30 based upon EM signals received from the trackers. The navigation computer 36 and/or the controller 30 may analyze the received EM signals to associate relative states thereto. Again, such navigation system 32 examples may have structural configurations that are different than the navigation system 32 configuration as shown throughout the Figures.

In yet another example, the navigation system 32 and/or localizer 44 utilize a machine vision system which includes a video camera coupled to the navigation computer 36. The video camera is configured to locate a physical object in a target space. The physical object has a geometry represented by virtual object data stored by the navigation computer 36. The detected objects may be tools, obstacles, anatomical features, trackers, or the like. The video camera and navigation computer 36 are configured to detect the physical objects using image processing techniques such as pattern, color, or shape recognition, edge detection, pixel analysis, neutral net or deep learning processing, optical character recognition, barcode detection, or the like. The navigation computer 36 can compare the captured images to the virtual object data to identify and track the objects. A tracker may or may not be coupled to the physical object. If trackers are utilized, the machine vision system may also include infrared detectors for tracking the trackers and comparing tracking data to machine vision data. Again, such navigation system 32 examples may have structural configurations that are different than the navigation system 32 configuration as shown throughout the Figures. Examples of machine vision tracking systems can be like that described in U.S. Pat. No. 9,603,665, entitled “Systems and Methods for Establishing Virtual Constraint Boundaries” and/or like that described in U.S. Provisional Patent Application No. 62/698,502, filed Jul. 16, 2018, entitled “Systems and Method for Image Based Registration and Calibration,” the entire contents of which are incorporated by reference herein.

The navigation system 32 and/or localizer 44 may have any other suitable components or structure not specifically recited herein. Furthermore, any of the techniques, methods, and/or components described above with respect to the camera-based navigation system 32 shown throughout the Figures may be implemented or provided for any of the other examples of the navigation system 32 described herein. For example, the navigation system 32 may utilize solely inertial tracking or any combination of tracking techniques.

As shown in FIG. 2, the controller 30 further includes software modules. The software modules may be part of a computer program or programs that operate on the manipulator computer 26, navigation computer 36, or a combination thereof, to process data to assist with control of the system 10. The software modules include instructions stored in one or more non-transitory computer readable medium or memory on the manipulator computer 26, navigation computer 36, or a combination thereof, to be executed by one or more processors of the computers 26, 36. Additionally, software modules for prompting and/or communicating with the operator may form part of the program or programs and may include instructions stored in memory on the manipulator computer 26, navigation computer 36, or a combination thereof. The operator interacts with the first and second input devices 40, 42 and the one or more displays 38 to communicate with the software modules. The user interface software may run on a separate device from the manipulator computer 26 and navigation computer 36.

The controller 30 includes a manipulator controller 60 for processing data to direct motion of the manipulator 14. In one example, as shown in FIG. 1, the manipulator controller 60 is implemented on the manipulator computer 26. The manipulator controller 60 may receive and process data from a single source or multiple sources. The controller 30 further includes a navigation controller 62 for communicating the state data relating to the anatomy to the manipulator 14 to the manipulator controller 60. The manipulator controller 60 receives and processes the state data provided by the navigation controller 62 to direct movement of the manipulator 14. In one example, as shown in FIG. 1, the navigation controller 62 is implemented on the navigation computer 36. The manipulator controller 60 or navigation controller 62 may also communicate states of the patient 12 and manipulator 14 to the operator by displaying an image of the anatomy and the manipulator 14 on the one or more displays 38. The manipulator computer 26 or navigation computer 36 may also command display of instructions or request information using the display 38 to interact with the operator and for directing the manipulator 14.

The one or more controllers 30, including the manipulator controller 60 and navigation controller 62, may be implemented on any suitable device or devices in the system 10, including, but not limited to, the manipulator computer 26, the navigation computer 36, and any combination thereof. As will be described herein, the controller 30 is not limited to one controller, but may include a plurality of controllers for various systems, components, or sub-systems of the surgical system 10. These controllers may be in communication with each other (e.g., directly, or indirectly), and/or with other components of the surgical system 10, such as via physical electrical connections (e.g., a tethered wire harness) and/or via one or more types of wireless communication (e.g., with a WiFi™ network, Bluetooth®, a radio network, and the like). Any of the one or more controllers 30 may be realized as or with various arrangements of computers, processors, control units, and the like, and may comprise discrete components or may be integrated (e.g., sharing hardware, software, inputs, outputs, and the like). Any of the one or more controllers may implement their respective functionality using hardware-only, software-only, or a combination of hardware and software. Examples of hardware include, but is not limited, single or multi-core processors, CPUs, GPUs, integrated circuits, microchips, or ASICs, digital signal processors, microcontrollers, field programmable gate arrays, systems on a chip, discrete circuitry, and/or other suitable hardware, and the like. The one or more controllers may implement software programs, software modules, algorithms, logical rules, look-up tables and other reference data, and various software layers for implementing any of the capabilities described herein. Equivalents of the software and hardware for the one or more controllers 30, and peripheral devices connected thereto, are fully contemplated.

As shown in FIG. 2, the controller 30 includes a boundary generator 66. The boundary generator 66 is a software module that may be implemented on the manipulator controller 60. Alternatively, the boundary generator 66 may be implemented on other components, such as the navigation controller 62. The boundary generator 66 generates virtual boundaries (VB) for constraining the tool holder 100, guide tube 101, and/or surgical tool. Such virtual boundaries (VB) may also be referred to as virtual meshes, virtual constraints, line haptics, or the like. The virtual boundaries (VB) may be defined with respect to a 3-D bone model registered to the one or more patient trackers 54, 56 such that the virtual boundaries (VB) are fixed relative to the bone model. The state of the tool holder 100, guide tube 101, and/or surgical tool is tracked relative to the virtual boundaries (VB). In one example, the state of the TCP is measured relative to the virtual boundaries (VB) for purposes of determining when and where haptic feedback force is applied to the manipulator 14, or more specifically, the tool 20 and/or bur 24.

A tool path generator 68 is another software module run by the controller 30, and more specifically, the manipulator controller 60. The tool path generator 68 generates a path for the tool holder 100 and/or surgical tool to traverse, such as for removing sections of the anatomy to receive an implant. One exemplary system and method for generating the tool path is explained in U.S. Pat. No. 9,119,655, entitled, “Surgical Manipulator Capable of Controlling a Surgical Instrument in Multiple Modes,” the disclosure of which is hereby incorporated by reference. In some examples, the virtual boundaries (VB) and/or tool paths may be generated offline rather than on the manipulator computer 26 or navigation computer 36. Thereafter, the virtual boundaries (VB) and/or tool paths may be utilized at runtime by the manipulator controller 60.

Additionally, it may be desirable to control the manipulator 14 in different modes of operation for the system 10. For example, the system 10 may enable the manipulator 14 to interact with the site using manual and semi-autonomous modes of operation. An example of the semi-autonomous mode is described in U.S. Pat. No. 9,119,655, entitled, “Surgical Manipulator Capable of Controlling a Surgical Instrument in Multiple Modes,” the disclosure of which is hereby incorporated by reference. In the semi-autonomous mode, the manipulator 14 directs movement of the tool holder 100 and, in turn, the surgical tool at the surgical site. In one instance, the controller 30 models the tool holder 100, guide tube 101, and/or surgical tool as a virtual rigid body and determines forces and torques to apply to the virtual rigid body to advance and constrain the tool holder 100, guide tube 101, and/or surgical tool along any trajectory or path in the semi-autonomous mode. Movement of the tool 20 in the semi-autonomous mode is constrained in relation to the virtual constraints generated by the boundary generator 66 and/or path generator 69,

In the semi-autonomous mode, the manipulator 14 is capable of moving the tool holder 100 free of operator assistance. Free of operator assistance may mean that an operator does not physically move the tool holder 100 by applying external force to move the tool holder 100. Instead, the operator may use some form of control to manage starting and stopping of movement. For example, the operator may hold down a button of a control to start movement of the tool holder 100 and release the button to stop movement of the tool holder 100. Alternatively, the operator may press a button to start movement of the tool holder 100 and press a button to stop motorized movement of the tool 20 along the trajectory or path. The manipulator 14 uses motorized movement to advance the tool holder 100 in accordance with pre-planned parameters.

Alternatively, the system 10 may be operated in the manual mode. Here, in one instance, the operator manually directs, and the manipulator 14 controls, movement of the tool holder 100. The operator physically contacts the tool holder 100 to cause movement of the tool holder 100. The manipulator 14 monitors the forces and torques placed on the tool holder 100 by the operator in order to position the tool holder. A sensor that is part of the manipulator 14, such as a force-torque transducer, measures these external forces and torques applied to the manipulator 14 and/or holder 100, e.g., in six degrees of freedom. In one example, the sensor is coupled between the distal-most link of the manipulator (J6) and the tool holder 100. In response to the applied forces and torques, the one or more controllers 30, 60, 62 are configured to determine a commanded position of the tool holder 100 by evaluating the forces/torques applied externally to the tool holder 100 with respect to virtual model of the tool holder 100 and/or surgical tool in a virtual simulation. The manipulator 14 then mechanically moves the tool holder 100 to the commanded position in a manner that emulates the movement that would have occurred based on the forces and torques applied externally by the operator. Movement of the tool holder 100 in the manual mode is also constrained in relation to the virtual constraints generated by the boundary generator 66 and/or path generator 69.

### II. Registration Instrument Overview

As previously stated, the guide tube 101 of the tool holder 100 may be temporarily fixed to a registration instrument 104, and the registration instrument 104 may support a tracker 106. FIGS. 3 and 6 illustrate two instances of a registration instrument 104 that may be temporarily fixed to the guide tube 101 and support the tracker 106.

a. First and Second Instances of the Registration Instrument

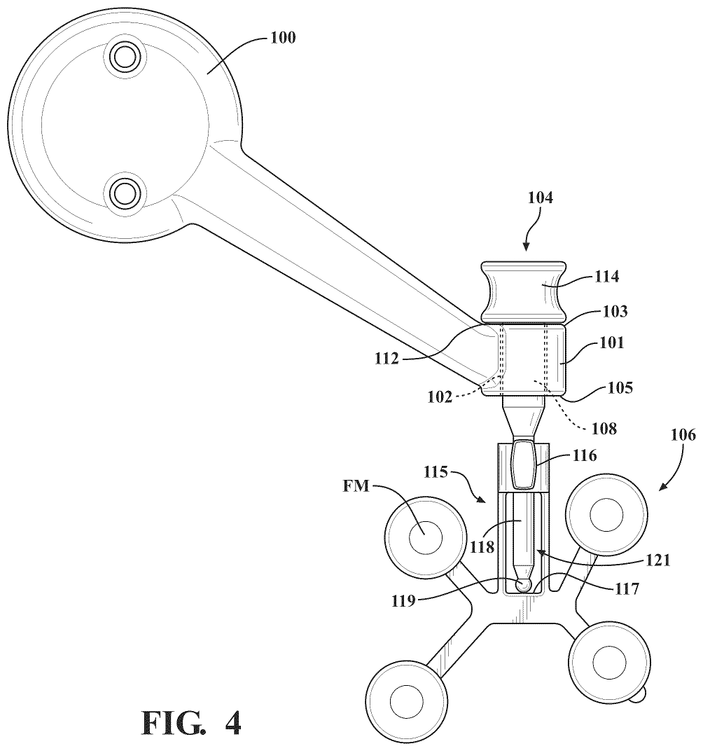

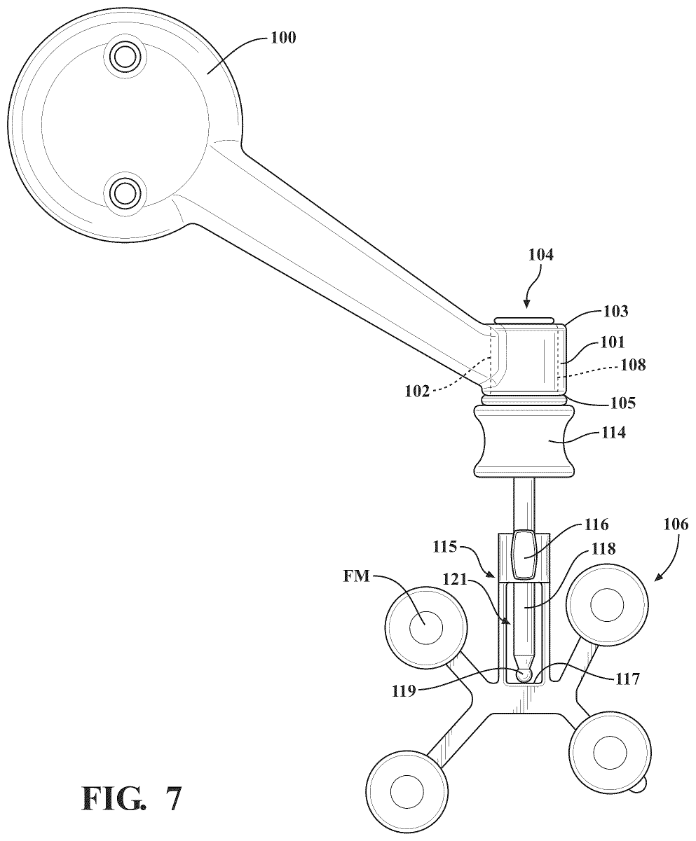

FIGS. 3 and 6 illustrate a first and second instance of the registration instrument 104, respectively. As shown in FIGS. 4 and 7, either instance of the registration instrument 104 may be temporarily fixed to the guide tube 101 through magnetic coupling. Additionally, each instance of the registration instrument 104 may support the tracker 106.

As shown in FIGS. 3 and 6, the first and second instances of the registration instrument 104 include a body 108. The registration instrument 104 may include a shaft 118 extending from the body 108 along the tool holder axis THA. Each of the first and second instances of the registration instrument 104 may also include a grip portion 114. As shown, the grip portion 114 is coupled to the body 108 and extends from the body 108 along the tool holder axis THA. Additionally, the body 108 may include a flange 112, and the grip portion 114 may define the flange 112.

The registration instrument 104 is configured to be inserted into the guide tube 101. As shown in FIG. 3, the first instance of the registration instrument 104 is configured to be inserted into the tool holder channel 102 along the tool holder axis THA and through the top end 103 of the guide tube 101. As shown in FIG. 6, the second instance of the registration instrument 104 is configured to be inserted into the tool holder channel 102 along the tool holder axis THA and through the bottom end 105 of the guide tube 101. Once the registration instrument 104 is inserted into the guide tube 101, the registration instrument 104 may be temporarily fixed to the guide tube 101 through magnetic coupling.

The body 108 of the registration instrument 104 is configured to be inserted into the tool holder channel 102 when the registration instrument 104 is temporarily fixed to the guide tube 101 through magnetic coupling. As shown in FIGS. 4 and 7, the body 108 of each of the first instance and the second instance of the registration instrument 104 is configured to be inserted into the tool holder channel 102. The body 108 may include a geometry (e.g., diameter) that substantially conforms to the geometry of the tool holder channel 102 to provide a snug fit between the body 108 and the tool holder channel 102, and to thereby constrain lateral movement between the body 108 and the guide tube 101, while allowing axial and rotational movement therebetween.

The grip portion 114 may be grasped by a user of the system 10 to facilitate insertion of the registration instrument 104 into the guide tube 101. For example, a user may grasp the grip portion 114 to insert the first instance of the registration instrument 104 through the top end 103 of the guide tube 101. As shown in FIG. 4, the grip portion 114 extends from the top end 103 of the guide tube 101 when the registration instrument 104 is temporarily fixed to the guide tube 101 through magnetic coupling. As another example, a user may grasp the grip portion 114 to insert the second instance of the registration instrument 104 through the bottom end 103 of the guide tube 101. As shown in FIG. 7, the grip portion 114 extends from the bottom end 105 of the guide tube 101 when the registration instrument 104 is temporarily fixed to the guide tube 101 through magnetic coupling.

The registration instrument 104 may freely rotate within the guide tube 101 while temporarily fixed to the guide tube 101 through magnetic coupling. For example, once the registration instrument 104 is magnetically coupled to the guide tube 101, the grip portion 114 may be manually rotated by a user of the system 10. Additionally, the tracker 106 may be supported by the shaft 118 such that the tracker 106 may rotate with rotation of the grip portion 114. Such a configuration allows a user to rotate the grip portion 114 to adjust a position of the tracker 106. For example, a user may rotate the grip portion 114 to adjust a position of the tracker 106 such that the fiducial markers FM face the localizer 44.

The flange 112 is configured to abut the guide tube 101 when the registration instrument 104 is temporarily fixed to the guide tube 101 through magnetic coupling. As shown in FIG. 4, the flange 112 of the first instance of the registration instrument 104 abuts and rests upon the top end 103 of the guide tube 101 when the registration instrument 104 is temporarily fixed to the guide tube 101 through magnetic coupling. As shown in FIG. 7, the flange 112 of the second instance of the registration instrument 104 abuts the bottom end 105 of the guide tube 101 when the registration instrument 104 is temporarily fixed to the guide tube 101 through magnetic coupling.

b. Magnetic Material of the First and Second Instances of the Registration Instrument

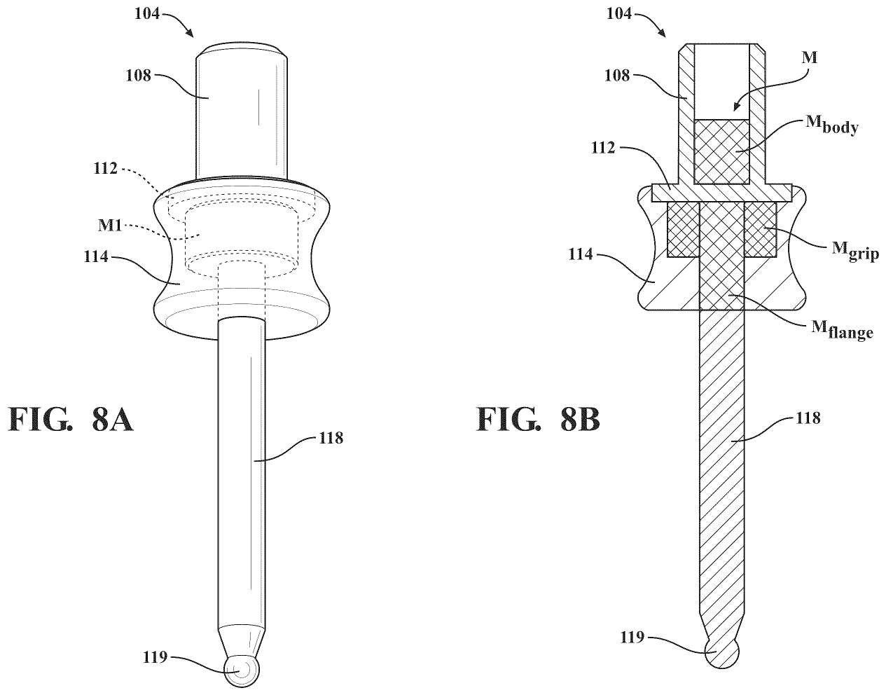

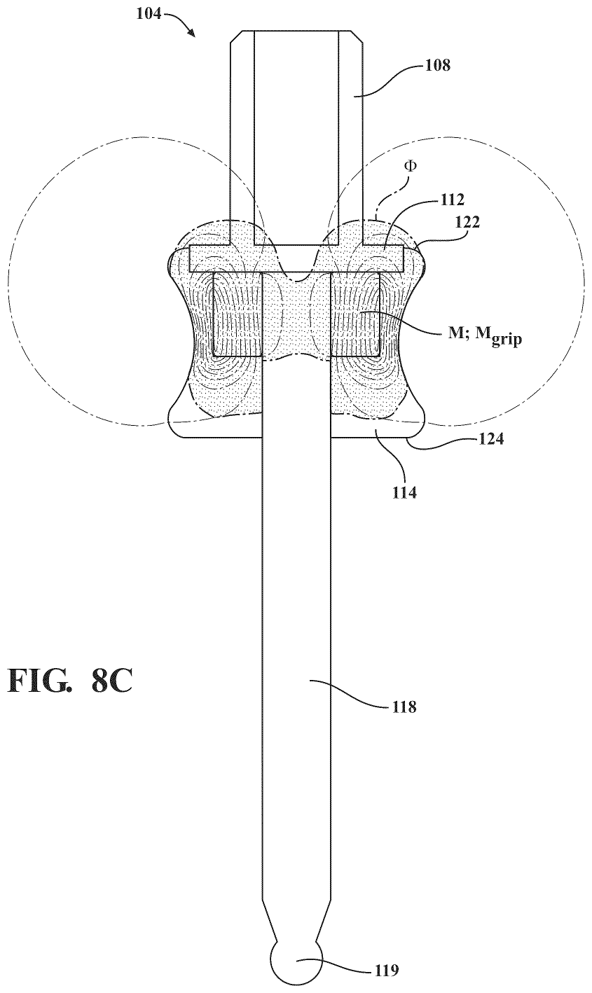

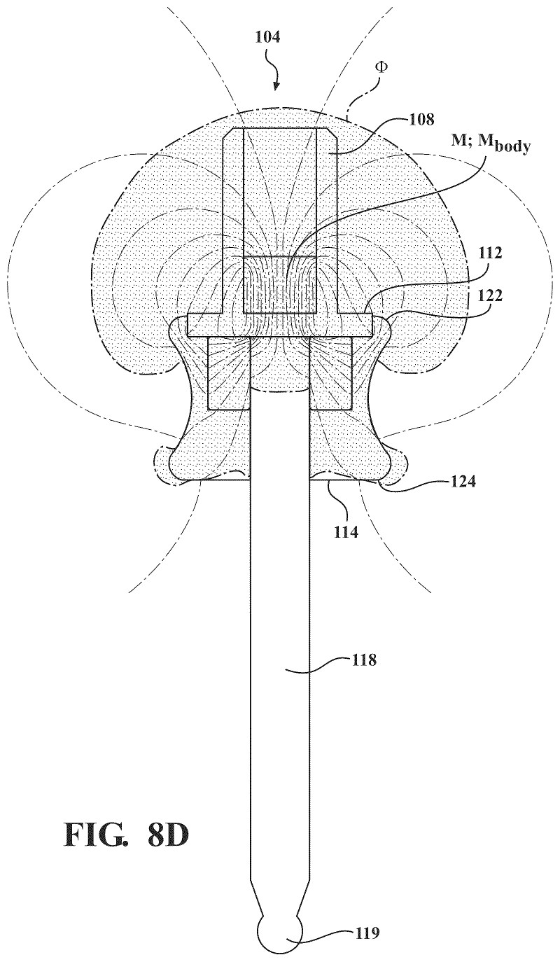

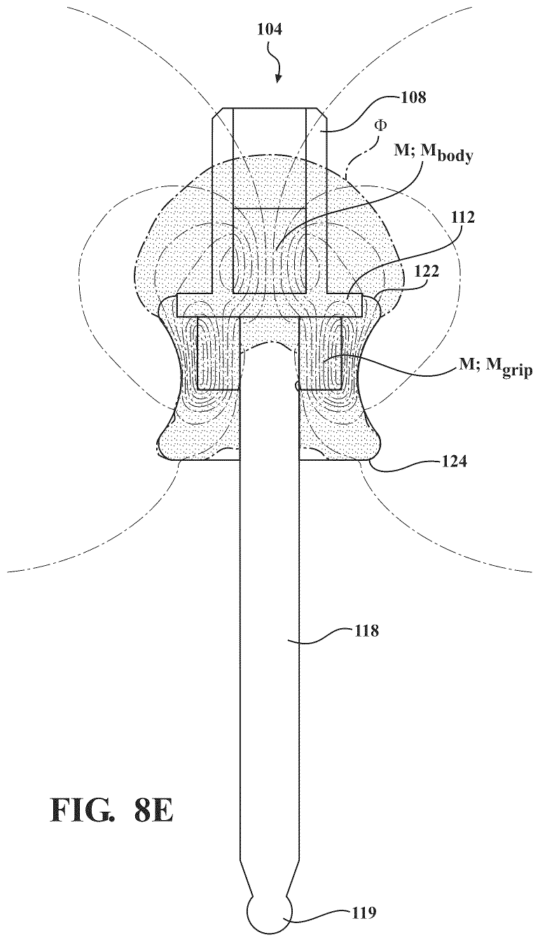

The registration instrument 104 is configured to temporarily fixed to the guide tube 101 through magnetically coupling. The first and second instances of the registration instrument 104 are shown as including magnetic material M in FIGS. 5A-5D and in FIGS. 8A-8E, respectively, the magnetic material M being configured to generate a magnetic field to magnetically couple the registration instrument 104 to the guide tube 101. Similarly, one or more of the guide tube 101, the guide tube wall 107, and/or the lobes 109 may be configured to generate a magnetic field to magnetically couple the registration instrument 104 to the guide tube 101.

Various components of the registration instrument 104 may include the magnetic material M. For example, the body 108 may include magnetic material M_{body}, the flange 112 may include the magnetic material M_{flange}, and the grip portion 114 may include magnetic material M_{grip}. The magnetic material M may include one or more of the magnetic materials M_{body}, M_{flange}, M_{grip}. In the instance of FIG. 5C, the grip portion 114 of the first instance of the registration instrument 104 includes the magnetic material M_{grip}. In the instance of FIG. 5D, the body 108 of the first instance of the registration instrument 104 includes the magnetic material M_{body}. In the instance of FIG. 8C, the grip portion 114 of the second instance of the registration instrument 104 includes the magnetic material M_{grip}. In the instance of FIG. 8D, the body 108 of the second instance of the registration instrument 104 includes the magnetic material M_{body}. In the instance of FIG. 8E, the grip portion 114 and the body 108 of the second instance of the registration instrument 104 includes the magnetic material M_{grip}, M_{body}, respectively, which form the magnetic material M of the second instance of the registration instrument 104.

In alternate instances, the magnetic material M may vary. For example, any component of the registration instrument 104 may include magnetic material M. For instance, the shaft 118 may optionally include magnetic material M. As another example, the magnetic material M may include any suitable size for magnetically coupling the registration instrument 104 to the guide tube 101. For example, the magnetic material M_{body} of FIG. 8E is of a greater size than the magnetic material M_{body} of FIG. 8D. Additionally, while the magnetic materials M_{body}, M_{flange}, M_{grip} are shown as separate components in FIGS. 5A-5D and 8A-8E, in other instances, two or more of the magnetic materials M_{body}, M_{flange}, M_{grip} may be combined as a single component.

Each of the magnetic materials M_{body}, M_{flange}, M_{grip} may magnetically couple a respective component of the registration instrument 104 to the guide tube 101 to temporarily fix the registration instrument 104 to the guide tube 101. For instance, the magnetic material M_{body} may magnetically couple the body 108 to the guide tube 101, the magnetic material M_{flange} may magnetically couple the flange 112 to the guide tube 101, and the magnetic material M_{grip} may magnetically couple the grip portion 114 to the guide tube 101.

In instances where the body 108 of the registration instrument 104 includes the magnetic material M_{body}, the magnetic material M_{body} magnetically couples to the guide tube 101 to temporarily fix the registration instrument 104 to the guide tube 101. For example, in the instance of FIG. 5D, the magnetic material M_{body} couples the body 108 of the first instance of the registration instrument 104 to the guide tube 101. In the instance of FIGS. 8D and 8E, the magnetic material M_{body} couples the body 108 of the second instance of the registration instrument 104 to the guide tube 101.

In instances where the flange 112 of the registration instrument 104 includes the magnetic material M_{flange}, the magnetic material M_{flange} is configured to magnetically couple the flange 112 to either the top end 103 or the bottom end 105 of the guide tube 101 to temporarily fix the registration instrument 104 to the guide tube 101. For example, in FIG. 4, the flange 112 of the first instance of the registration instrument 104 rests upon and abuts the top end 103 of the guide tube 101. As follows, in instances where the flange 112 of the first instance of the registration instrument 104 includes the magnetic material M_{flange}, the magnetic material M_{flange} may magnetically couple the flange 112 to the top end 103 of the guide tube 101. In FIG. 7, the flange 112 of the second instance of the registration instrument 104 abuts the bottom end 105 of the guide tube 101. As follows, in instances where the flange 112 of the second instance of the registration instrument 104 includes the magnetic material M_{flange}, the magnetic material M_{flange} may magnetically couple the flange 112 to the bottom end 105 of the guide tube 101.

In instances where the grip portion 114 of the registration instrument 104 includes the magnetic material M_{grip}, the magnetic material M_{grip} is configured to magnetically couple the grip portion 114 to either the top end 103 or the bottom end 105 of the guide tube 101. For example, in the instance of FIG. 5C, the magnetic material M_{grip} couples the grip portion 114 of the first instance of the registration instrument 104 to the guide tube 101. In the instance of FIGS. 8C and 8E, the magnetic material M_{body} couples the grip portion 114 of the second instance of the registration instrument 104 to the guide tube 101.

The magnetic material M may include any magnetic material suitable for magnetically coupling the registration instrument 104 to the guide tube 101. For example, the magnetic material M may include any suitable ferrous magnetic metal, such as 17-4 stainless steel and/or 455 series stainless steel.

The guide tube 101, the guide tube wall 107, and/or the lobes 109 may include any magnetic material suitable for magnetically coupling the registration instrument 104 to the guide tube 101. For example, the guide tube 101, the tool holder channel 102, the guide tube wall 107, and/or the lobes 109 may include any suitable ferrous magnetic metal, such as 17-4 stainless steel and/or 400 stainless steel.

Components of the registration instrument 104 that are not configured to generate a magnetic field, i.e. non-magnetic components of the registration instrument 104, may include non-ferrous material. For example, any component of the registration instrument 104 other than the magnetic material M may include non-ferrous material, such as 300 series stainless steel and/or plastic.

The non-ferrous material of non-magnetic components of the registration instrument 104 may be configured to direct the magnetic flux field Φ. Advantageously, by directing the magnetic flux field @, the non-ferrous material of the non-magnetic components limits inadvertent magnetic coupling of magnetic objects near the registration instrument 104. As an example, in the instance of FIG. 5C, the grip portion 114 includes non-ferrous material such that the magnetic flux field Φ generated by the magnetic material M_{grip} is directed away from a top end 122 of the grip portion 114 and toward a bottom end 124 of the grip portion 114. In the instance of FIG. 5D, the grip portion 114 includes non-ferrous material such that the magnetic flux field Φ generated by the magnetic material M_{body} is directed away from the top end 122 of the grip portion 114 and toward the bottom end 124 of the grip portion 114. In the instance of FIG. 8C, the grip portion 114 includes non-ferrous material such that the magnetic flux field Φ generated by the magnetic material M_{grip} is directed away from the bottom end 124 of the grip portion 114 and toward the top end 122 of the grip portion 114. In the instance of FIG. 8D, the grip portion 114 includes non-ferrous material such that the magnetic flux field Φ generated by the magnetic material M_{body} is directed away from the bottom end 124 of the grip portion 114 and toward the top end 122 of the grip portion 114. In the instance of FIG. 8E, the grip portion 114 includes non-ferrous material such that the magnetic flux field Φ generated by the magnetic materials M_{body}, M_{grip} is directed away from the bottom end 124 of the grip portion 114 and toward the top end 122 of the grip portion 114.

c. Supporting a Tracker with the Registration Instrument

The registration instrument 104 may support the tracker 106. For example, the body 108 may be configured to support the tracker 106. In instances where the registration instrument 104 includes the shaft 118, such as the instances of FIGS. 3-4 and 6-7, the shaft 118 may be configured to support the tracker 106.

The tracker 106 is configured to be removably attached to the shaft 118. In the first instance shown in FIGS. 3-4, the tracker 106 may be secured to the registration instrument 104 once the guide tube 101 has received the registration instrument 104. In the second instance shown in FIGS. 6-7, the tracker 106 may be secured to the registration instrument 104 prior to or after the guide tube 101 has received the registration instrument 104.

As shown in FIGS. 3-4 and 6-7, the tracker 106 may include a coupling interface 115 configured to be installed onto and secured to the shaft 118. As shown, the coupling interface 115 may include a receptacle 121 configured to receive a portion of the shaft 118 and a securing mechanism 120 configured to secure the tracker 106 to the shaft 118. Referring to FIGS. 3 and 6, the securing mechanism 120 is disposed perpendicular to the tool holder axis THA. The securing mechanism 120 may be manipulated to apply force to the shaft 118 to secure the tracker 106 to the shaft 118. For example, in the instance of FIGS. 3 and 6, the securing mechanism 120 may be manipulated by a knob 116.

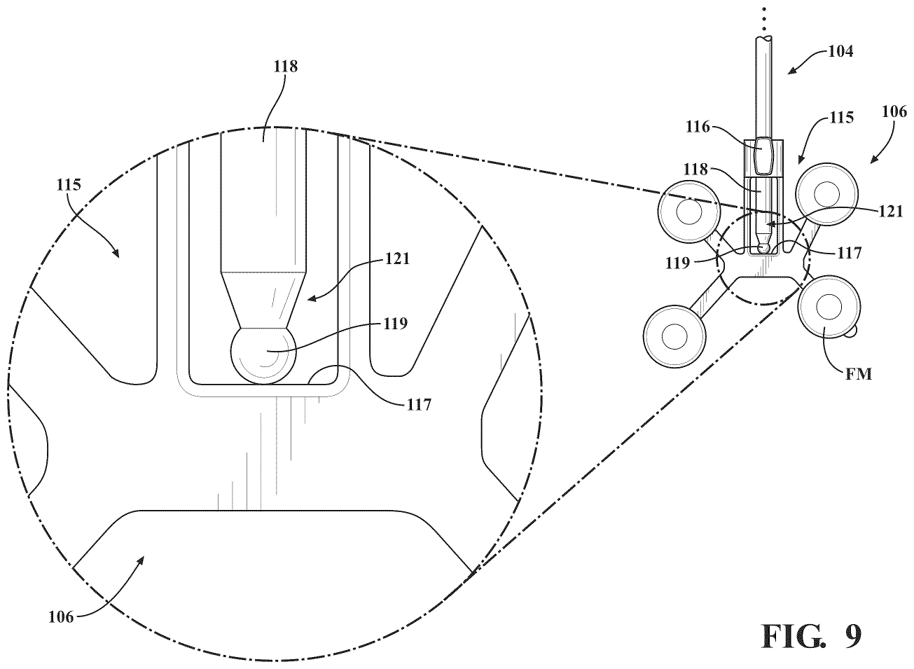

As shown in FIG. 9, the shaft 118 may include a reference tip 119 and the coupling interface 115 may include a reference surface 117. When the tracker 106 is removably attached to the shaft 118, a reference tip 119 of the shaft 118 is configured to abut the reference surface 117 of the coupling interface 115. As will be explained in greater detail below, contact between the reference tip 119 and the reference surface 117 fixes a location of the tracker 106 relative to the registration instrument 104 during registration of the robotic manipulator 14.

Other instances of the tracker 106 are contemplated. In some instances, the tracker 106 may be integrally formed with the registration instrument 104. For example, the tracker 106 may be integrally fixed to the shaft 118. In such an instance, the reference instrument 104 may optionally omit the reference tip 119 and the coupling interface 115 may optionally omit the receptacle 121 and the reference surface 117. In some instances, the tracker 106 may be configured to be supported by a component of the registration instrument 104 other than the shaft 118. For example, the tracker 106 may be coupled to or integrally formed with the body 108, the flange 112, or the grip portion 114 of the registration instrument 104. The tracker 106 may also be coupled to or integrally formed with the guide tube 101. In some instances, the tracker 106 may be omitted and the fiducial markers FM may be coupled to or integrally formed with a component of the registration instrument 104 and/or the guide tube 101. Additionally, the fiducial marker FM may include any suitable shape. For example, the fiducial marker FM may include a cuboidal or spherical shape.

### III. Registration of the Surgical System

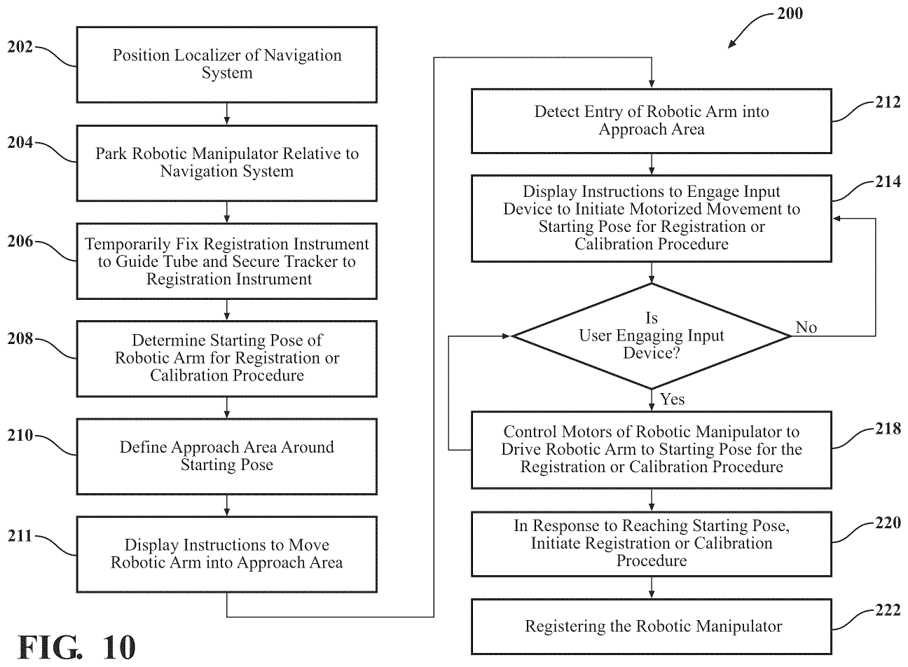

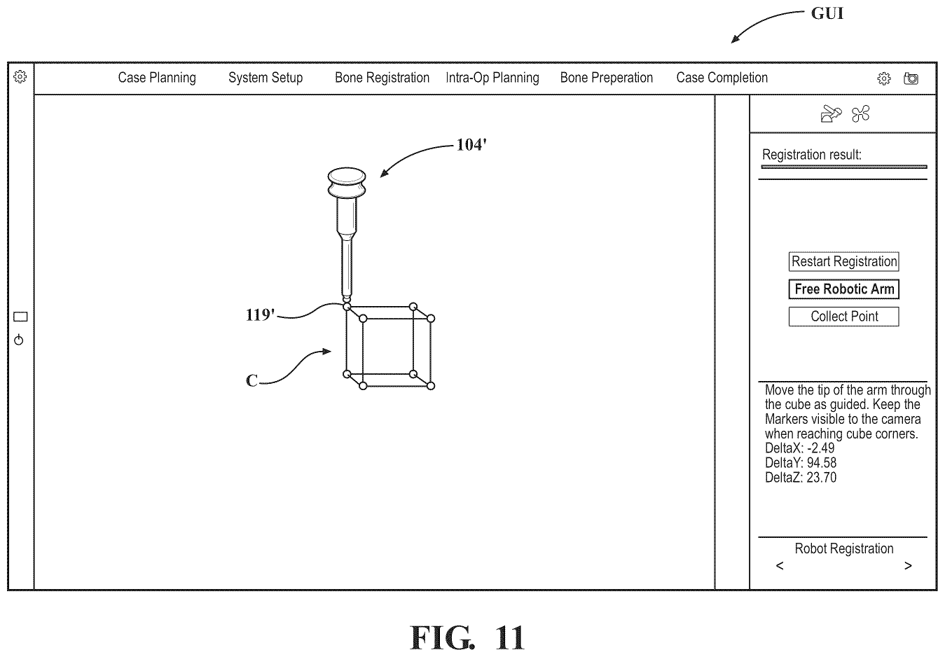

Referring now to FIG. 10, an example method 200 for providing a motorized movement of the robotic arm 18A to a starting pose for a registration or calibration routine for the robotic arm 18A is shown. The method 200 may be executed using the controllers 30 described above, and reference thereto is made in the description of the method 200. Additionally, the method 200 may be like that described in U.S. patent application Ser. No. 17/513,324, entitled “Robotic Surgical System with Motorized Movement to a Starting Pose for a Registration or Calibration Routine”, which is incorporated herein by reference.

At step 202, the localizer 44 of the navigation system 32 is positioned, for example in an operating room. For example, the cart assembly 34 may be set (parked, locked, braked, fixed, etc.) in the position where it will preferably stay for a duration of the surgical procedure. In some instances, the navigation system 32 is configured to provide, via the display 38, instructions for positioning the localizer 44 of the navigation system 32.

At step 204, the manipulator 14 and the navigation system 32 are positioned and parked relative to one another, for example such that the manipulator 14 and the localizer 44 of the navigation system 32 are separated by less than or equal to a preset distance. For example, the base 16 can be rolled, steered, etc. into a desired position relative to the navigation system 32 and relative to other structures in the operating room (e.g., relative to a table/bed on which a patient can be positioned during a surgical procedure). As another example, the base 16 could be parked first and the navigation system 32 (e.g., the localizer 44 of the navigation system 32) can be moved toward the base 16. In some cases, the base 16 is positioned such that the patient will be located between the base 16 and the localizer 44 of the navigation system 32. In some instances, the navigation system 32 is configured to provide, via a display 38, instructions for positioning the localizer 44 of the navigation system 32. In some cases, the navigation system 32 is used to provide live updates of the position of the base 16 relative to a target parking position displayed on the display 38. Accordingly, the base 16 can be guided to a parking position relative to other components used in the operating room.

At step 206, the registration instrument 104 is temporarily fixed to instrument to the guide tube 101, and the tracker 106 is secured to the registration instrument 104. As described above, the registration instrument 104 may be temporarily fixed to the guide tube 101 through magnetic coupling. For example, the first instance of the registration instrument 104 shown in FIGS. 3 and 4 may be inserted into the guide tube 101 and the tracker 106 may be removably attached to the shaft 118 of the registration instrument 104. As another example, the tracker 106 may be removably attached to the shaft 118 of the second instance of the registration instrument 104 before or after the registration instrument 104 is inserted into the guide tube 101. The tracker 106 is removably attached to the shaft 118 such that the reference tip 119 of the shaft 118 contacts the reference surface 117 of the tracker 106, the fiducial markers FM of the tracker 106 are properly positioned for tracking by the localizer 44.

At step 208, a starting pose of the robotic arm 18A for a registration or calibration routine is determined. The starting pose may be associated with an expected position of a surgical field in which a surgical procedure will be performed using a surgical tool attached to the robotic arm 18A. For example, the starting pose may be representative of cutting poses that will be used during the surgical procedure. In some instances, the controller 30 determines the starting pose based on relative positions of the localizer 44 and the base 16 of the manipulator 14. For example, the starting pose may be determined to ensure or improve the likelihood that the tracker 106 remains within the line-of-sight of the localizer 44 of the navigation system 32 throughout the calibration and registration procedures. In some instances, the starting pose is automatically calculated based on one or more of these criteria each time the method 200 is performed (e.g., for each surgical operation). In other instances, the starting pose is predetermined or preprogrammed based on the various criteria, for example such that properly parking the base 16 in an acceptable position ensures that the starting pose will be properly situated in the operating room.

In some instances of step 208, the starting pose for registration or calibration is determined by performing an optimization process to find a best working volume for cuts in a total knee arthroplasty procedure (or other procedure in other applications). The optimization process may consider factors such as estimated calibration error for the robotic arm, anthropomorphic models of the surgeon/user relating to usability and ergonomics, surgeon height, surgeon preferences, probable position of the patient on the table, and other operating room constraints. The determination may be made using an assumption that the camera is positioned across the knee from the manipulator 14. The starting pose may be selected as the center of the optimized working volume. In some instances of step 208, the starting pose is selected to corresponding to a working volume where the robotic arm 18A has a lowest calibration error and estimated error due to compliance in the arm during use. Additionally, the starting pose may be selected such that motorized alignment ends in a plane that is parallel to the expected orientation of the camera unit 46 of the navigation system 32.

Optionally, at step 210, an approach area may be defined around the starting pose. The approach area defines a space in which motorized movement of the robotic arm to the starting pose can be initiated as described below with reference to steps 212-218. In some instances, the approach area is defined by a virtual boundary, for example a sphere centered on the starting pose. In some instances, the approach area is defined in a coordinate system of the navigation system 32. In some instances, the approach area is defined in terms of joint angles of the robotic arm 18A.

The approach area may be defined in various ways in various instances. For example, in some instances the approach area is defined to balance multiple considerations. Reducing a size of the approach area can reduce a risk of the robotic arm 18A colliding with objects or people in the operating room motorized movement. Also, determination of the approach area can include ensuring that the approach area is sufficiently large to enable a user to easily move the registration instrument 104 in the approach area. The approach area can also be defined to ensure that it is consistent with the range of the robotic arm so that the robotic arm is capable of reaching the approach area. The approach area can also be sized and positioned based on a preferred distance and speed for the motorized motion in later steps, i.e., such that the robotic arm enters the approach area at a location which is within an acceptable distance of the starting pose for the registration or calibration procedure and from which the motorized motion can be performed in an acceptable amount of time (e.g., less than a threshold duration) and at an acceptable velocity (e.g., less than a threshold velocity). The approach area may vary based on whether the procedure is to be performed on a right or left side of the patient's body (e.g., right knee vs. left knee).

At step 211, instructions are displayed which instruct a user to move the robotic arm into the approach area. For example, the navigation controller 62 can cause the display 38 to display a graphical user interface including a graphic that illustrates movement of the robotic arm 18A into the approach area. The graphical user interface may also include text-based instructions.

At step 212, entry of the robotic arm 18A into the approach area is detected. The robotic arm 18A can be moved into the approach area manually by a user. That is, the user can exert a force on the robotic arm 18A to push the robotic arm into the approach area. In some instances, detecting entry of the robotic arm 18A into the approach area includes tracking the fiducial markers FM of the tracker 106 with the localizer 44 and determining whether the distal end of the robotic arm 18A is in an approach area defined in a coordinate system used by the navigation system 32. In other instances, detecting entry of the robotic arm 18A includes checking joint angles of the robotic arm 18A (e.g., from encoders at the joints) against one or more criteria which define the approach area in terms of joint angles of the robotic arm 18A. In such instances, detecting entry of the robotic arm 18A into the approach area can be performed independently of the navigation system 32. Thus, step 212 corresponds to determining that the robotic arm 18A is in a position from which it can be automatically moved to the starting pose determined in step 208.

At step 214, instructions are displayed which instruct a user to activate (e.g., engage, disengage, depress, release, etc.) an input device or otherwise input a command to initiate motorized movement of the robotic arm 18A to the starting pose for the registration or calibration routine. For example, the navigation controller 62 may cause the display 38 to display a graphical user interface that includes a graphic showing a user engaging an input device, for example depressing a trigger, depressing a foot pedal, or otherwise engaging some other input device (e.g., mouse, button, pedal, trigger, switch, sensor). As another example, a microphone may be communicable with the navigation controller 62 such that a voice command can be used to initiate motorized movement. As another example, touchless gesture control could be used, for example using a machine vision approach, to provide a command to initiate automated alignment. As another example, the command can be input by moving the registration instrument 104 in a particular direction. The command can be provided by a primary user (e.g., surgeon) in the sterile field and/or by a second person, for example a technician or nurse elsewhere in the operating room.

Accordingly, in step 214, an option is provided for the user to initiate motorized movement of the robotic arm 18A to the starting pose for the registration or calibration routine. In alternative instances, steps 214 and 216 are omitted and motorized movement is automatically initiated when the robotic arm 18A enters the approach area without additional input from a user.

At step 216, a determination is made of whether the user is still activating the input device as instructed in step 214. For example, engagement of the input device (e.g., depression of a trigger) may create an electrical signal from the input device to the navigation controller 62. In such an example, the controller 30 can determine whether the user is activating the input device based on whether the electrical signal is received. For example, presence of the signal from the input device may cause the controller 30 to determine at step 216 that the user is engaging the input device, whereas absence of the signal from the input device may cause the controller 30 to determine at step 216 that the user is not engage the input device.

If a determination is made at step 216 that the user is not activating the input device (i.e., “No” at step 216 in FIG. 10) (i.e., deactivation of the input device, for example by engagement or disengagement of an input device), the method 200 returns to step 214 to continue to display instructions to the user to engage the input device to initiate motorized movement to the starting pose. In some instances, an audible, haptic, or other alert may provide if the user does not engage the input device after a certain amount of time or according to some other criteria that indicates that the user is not aware of the instructions to engage the input device to initiate motorized movement to the starting pose.

If a determination is made at step 216 that the user is engaging the input device (i.e., “Yes” at step 216 in FIG. 10), the method 200 moves to step 218 where motors of the robotic arm 18A are controlled to drive the robotic arm to the starting pose for the registration or calibration routine. That is, in step 218 the manipulator 14 is controlled to provide motorized movement of the robotic arm 18A from a pose where the user first engages the input device to the starting pose for a registration or calibration routine identified in step 208. In some instances, motorized movement is performed along a shortest/straight path to the starting pose. In some instances, step 218 includes automatically planning a path between an initial position and the starting poses for the registration or calibration routine, and then control the robotic arm to provide movement along the planned path. The path can be straight or curved. In some instances, the path is planned such that motorized movement of the robotic arm 18A in step 218 will take between a lower duration threshold and an upper duration threshold (e.g., between approximately 4 seconds and approximately six seconds).

Motorized movement of the robotic arm 18A to the starting pose in step 218 can includes movement in one to six degrees of freedom, for example including moving a distal end of the robotic arm 18A to a location identified by the starting pose and providing rotations to align with an orientation identified by the starting pose. In some instances, motorized movement includes arranging joint angles of the robotic arm 18A in a preferred (e.g., predefined) arrangement, for example an arrangement that facilitate calibration, registration, and/or completion of the surgical procedure. In other instances, for example for a seven degree of freedom robot, motorized movement can be performed such that the target starting position of the registration instrument 104 is defined and used for control without regards to angles or other positions of the arm 18A.

As illustrated in FIG. 10, the controller 30 can continue to make the determination in step 216 of whether the user is engaging the input device. In some scenarios, the user will engage the input device to initiate motorized movement, but then disengage from the input device before the motorized movement has resulted in arrival at the starting pose for the registration or calibration routine. In such scenarios, and in some instances, the controller 30 determines in step 216 that the user is no longer engaging the input device and stops the motorized movement of the robotic arm. Method 200 can then return to step 214, where a user is instructed to restart motorized movement by reengaging the input device.

If the user continues to engage the input device, motorized movement continues until the robotic arm 18A reaches the starting pose for the registration or calibration routine. At step 220, in response to reaching the starting pose, a registration or calibration routine is initiated. Initiating the registration or calibration routine can include starting one or more data collection processes, for example tracking of an end effector array and base array by the navigation system 32, any other tracking of the manipulator 14, controlling the robotic arm 18A to provide additional motorized movements or to constrain manual movement of the robotic arm 18A, and/or providing instructions for user actions to support the registration or calibration routine via the display 38.

For example, the registration or calibration routine may be provided on a graphical user interface, such as the graphical user interface GUI shown in FIG. 11. The graphical user interface GUI may be displayed on the display 38 in response to the robotic arm 18A reaching the starting pose for the registration or calibration routine. As shown, the registration or calibration routine instructs the user to manually move the registration instrument 104 coupled to the robotic arm 18A. Specifically, the graphical user interface GUI generates a virtual representation 104′ of the registration instrument 104 based on tracking the tracker 106. For instance, as the registration instrument 104 is moved by the user, the localizer 44 tracks the movement of the tracker 106 and the graphical user interface GUI updates a location of the virtual representation 104′ of the registration instrument 104 based on the tracking data of the tracker 106. Additionally, the graphical user interface GUI generates a cube C with vertices. The registration or calibration routine instructs the user to move the registration instrument 104 such that the virtual representation 104′ of the registration instrument 104 is moved to the vertices of a cube, while the tracker 106 is in view of the localizer 44.

In the example of FIG. 11, the registration or calibration routine provides instructions to a user to cause the user to manually move the registration instrument 104 coupled to the robotic arm 18A to the vertices of a cube C. The cube C in such an example is located proximate the starting pose for the registration or calibration routine identified in step 208, for example centered on the starting point or having a first vertex at the starting pose. The motorized movement to the starting pose can be seen as guiding the registration instrument 104 and/or tracker 106 to the cube C. Geometries other than a cube can be used in other instances, for example, a geometry may be selected such that each joint J1-J6 of the arm 18A is exercised during the registration or calibration routine. Additionally, the graphical user interface provided on the display 38 is further described in U.S. patent application Ser. No. 17/513,324, entitled “Robotic Surgical System with Motorized Movement to a Starting Pose for a Registration or Calibration Routine”, which is incorporated herein by reference.

Once the registration or calibration routine has been initialized in step 220, the method 200 may proceed to the step 222 of registering the robotic manipulator 14 to the localizer coordinate system LCLZ. During step 222, the controller 30 may facilitate control of the robotic manipulator 14 for moving the tracker 106 in various positions. As the tracker 106 is moved to various positions and is tracked by the localizer 44, the navigation controller 62 is able to register the robotic manipulator 14 to the localizer coordinate system LCLZ by comparing tracking data related to the tracker 106 with kinematic data related to the robotic manipulator 14. For example, the navigation controller 62 may obtain tracking data related to the tracker 106 in the various poses from the localizer 44 and kinematic data related to the robotic manipulator 14 in the various poses from the robotic manipulator 14 (e.g., from the manipulator controller 60). Once the navigation controller 62 receives the tracking data and the kinematic data, the navigation controller 62 may compare the tracking data and the kinematic data for defining a relationship between the manipulator coordinate system MNPL and the localizer coordinate system LCLZ to register the robotic manipulator 14.

As previously stated, contact between the reference tip 119 and the reference surface 117 fixes a location of the tracker 106 relative to the registration instrument 104. By fixing the location of the tracker 106 relative to the registration instrument 104 and, furthermore, the guide tube 101, the navigation controller 62 is able to determine a position or pose of the robotic manipulator 14 in the localizer coordinate system LCLZ based on tracking data received from the localizer. Once the navigation controller 62 receives the kinematic data related to the robotic manipulator 14, the navigation controller 62 is also able to determine a position or pose of the robotic manipulator 14 in the manipulator coordinate system MNPL. By comparing the position or pose of the robotic manipulator 14 in the localizer coordinate system LCLZ and the position or pose of the robotic manipulator 14 in the manipulator coordinate system MNPL, the navigation controller 62 is able to define the relationship between the manipulator coordinate system MNPL and the localizer coordinate system LCLZ to register the robotic manipulator 14.

Although specific features of various embodiments of the disclosure may be shown in some drawings and not in others, this is for convenience only. In accordance with the principles of the disclosure, any feature of a drawing or other embodiment may be referenced and/or claimed in combination with any feature of any other drawing or embodiment.

This written description uses examples to describe embodiments of the disclosure and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A surgical system comprising:
a robotic manipulator supporting a guide tube; and
an instrument configured to be temporarily fixed to the guide tube through magnetic coupling; and
a tracker configured to be supported by the instrument.

2. The surgical system of claim 1, wherein:
the guide tube includes a top end and bottom end;
the guide tube defines a channel extending between the top end and the bottom end; and
the instrument comprises a body configured to be inserted into the channel when the instrument is temporarily fixed to the guide tube through magnetic coupling.

3. The surgical system of claim 2, wherein the body comprises magnetic material configured to magnetically couple to the guide tube.

4. The surgical system of claim 2, wherein the body comprises a flange configured to abut either the top end or the bottom end of the guide tube when the instrument is temporarily fixed to the guide tube through magnetic coupling.

5. The surgical system of claim 4, wherein the flange comprises magnetic material configured to magnetically couple the flange to either the top end or the bottom end of the guide tube.

6. The surgical system of claim 4, wherein the instrument is configured to be inserted into the channel through the top end of the guide tube, and wherein the flange is configured to abut and rest upon the top end of the guide tube when the instrument is temporarily fixed to the guide tube through magnetic coupling.

7. The surgical system of claim 4, wherein the instrument is configured to be inserted into the channel through the bottom end of the guide tube, and wherein the flange is configured to abut the bottom end of the guide tube when the instrument is temporarily fixed to the guide tube through magnetic coupling.

8. The surgical system of claim 4, wherein the instrument includes a grip portion coupled to the body and being configured to extend from the guide tube when the instrument is temporarily fixed to the guide tube through magnetic coupling.

9. The surgical system of claim 8, wherein the grip portion defines the flange.

10. The surgical system of claim 8, wherein the grip portion comprises magnetic material configured to magnetically couple the grip portion to either the top end or the bottom end of the guide tube.

11. The surgical system of claim 8, wherein the instrument is configured to be inserted into the channel through the top end of the guide tube, and wherein the grip portion is configured to extend from the top end of the guide tube when the instrument is temporarily fixed to the guide tube through magnetic coupling.

12. The surgical system of claim 8, wherein the instrument is configured to be inserted into the channel through the bottom end of the guide tube, and wherein the grip portion is configured to extend from the bottom end of the guide tube when the instrument is temporarily fixed to the guide tube through magnetic coupling.

13. The surgical system of any claim 2, wherein the instrument comprises a shaft that extends from the body, and wherein the tracker is configured to be supported by the shaft.

14. The surgical system of claim 13, wherein:
the tracker is configured to be removably attached to the shaft; and
the tracker comprises a coupling interface configured to be installed onto and secured to the shaft.

15. The surgical system of claim 14, wherein:
the shaft includes a reference tip located at a distal end of the shaft; and
the coupling interface comprises a reference surface configured to abut the reference tip of the shaft.

16. The surgical system of claim 14, wherein the coupling interface supports a securing mechanism disposed perpendicular to an axis of the shaft and the securing mechanism is configured to be manipulated to apply force to the shaft to secure the tracker to the shaft.

17. A surgical instrument for use with a robotic manipulator that supports a guide tube, the surgical instrument comprising:
a body configured to be temporarily fixed to the guide tube through magnetic coupling and the body being configured to support a tracker.

18. The surgical instrument of claim 17, wherein the guide tube includes a top end and bottom end and the guide tube defines a channel extending between the top end and the bottom end, and wherein:
the body is configured to be inserted into the channel;
the body comprises a flange configured to abut either the top end or the bottom end of the guide tube; and
at least one of the body and the flange comprises magnetic material to magnetically couple to the guide tube.

19. The surgical instrument of claim 18, further comprising a grip portion coupled to the body, wherein the grip portion defines the flange, and wherein the grip portion comprises a magnetic material configured to magnetically couple to the guide tube.

20. The surgical instrument of claim 17, comprising a shaft extending from the body, the shaft including a reference tip located at a distal end of the shaft, wherein the tracker comprises a coupling interface comprising a reference surface, the coupling interface configured to be installed onto and secured to the shaft to removably attach the tracker to the shaft, the reference surface being configured to abut the reference tip of the shaft.
